(19) United States
(12) Patent Application Publication
(10) Pub. No.: US 2008/0287662 A1
(43) Pub. Date: Nov. 20, 2008

(54) Balanites Aegyptiaca Saponins and Uses Thereof

(76) Inventor: Zeev Wiesman, Lehavim (IL)

Correspondence Address: BROWDY AND NEIMARK, P.L.L.C.;624 NINTH STREET, NW, SUITE 300, WASHINGTON, DC, 20001-5303, US

(21) Appl. No.: 11/993,899

(22) PCT Filed: Jun. 22, 2006
(86) PCT No.: PCT/IL2006/000733

§ 371 Date: May 27, 2008

Related U.S. Application Data
(60) Provisional application No. 60/692,661, filed on Jun. 22, 2005.
(60) Provisional application No. 60/781,332, filed on Mar. 13, 2006.

(51) Int. Cl. C07H 15/00

(52) U.S. Cl. 536/4.1

(57) ABSTRACT

The invention relates to stable preparations of saponin nanovesicles, which can encapsulate active biological or chemical materials such as drugs, pesticides, vitamins, minerals and nutrients, and protect them. The preparations can be used, for example, in agricultural, pesticidal, dermatological and cosmetic compositions. The saponins per se can further be used as adjuvants for irrigation water. The invention further relates to novel saponins extracted from Balanites aegyptiaca.

### FIELD OF THE INVENTION

The present invention relates to saponins extracted from Balanites aegyptiaca trees as well as to applications of these and other saponins.

### BACKGROUND OF THE INVENTION

Balanites aegyptiaca Del. (Zygophyllaceae), popularly known as “desert date”, is a widely grown desert tree with a multitude of potential uses. It is found throughout the Sudano-Sahelian region of Africa and in other arid areas of Africa, the Middle East, India and Burma. It is one of the most drought-resistance tree species in these arid regions. In Israel, Balanites trees are found in Ein-Gedi oasis, in the Aiava and in the Bet-Shean valley, considered to be the northern limit of Balanites population

Plant tissues from B. aegyptiaca have been used in a variety of folk medicines in Africa and Asia. Extracts from several parts of this tree have been intensively used in Africa and India for various ethnobotanical purposes. For example, Balanites extracts were shown to exhibit antifeedant, antidiabetic, molluscicide, anthelmintic and contraceptive activities (Jain and Tripathi, 1991; Kamel et al., 1991; Liu and Nakanishi, 1982; Ibrahim, 1992; Rao et al., 1997). Earlier studies have shown that B. aegyptiaca contains steroidal saponins. Most of these studies have reported that the presence of saponins is the main cause behind these activities. Besides its medicinal uses, Balanites trees are widely used as fodder, and for timber purposes.

The fruit of B. aegyptiaca consists of an epicarp (5-9%), a mesocarp (28-33%), an endocarp (49-54%) and a kernel (8-12%). The oil-rich kernel is used by the local people as a source of edible oil. Although B. aegyptiaca has been used for many purposes—from ethnobotanicals to firewood, from forage to edible fruit, this plant is considered one of the most neglected species of arid regions and has yet to be domesticated.

Saponins, often referred to as a “natural detergent” because of their foamy texture, are a class of complex glycosides mainly found in a variety of higher plants as secondary metabolites. Saponins possess a variety of bioactive qualities: they are cytotoxic, antifungal, antimicrobial, anti-inflammatory, etc. Saponins are also considered to be the major effective component of many traditional medicines. Saponins are amphiphilic molecules consisting of a hydrophobic aglycone linked to one or more hydrophilic sugar moieties. Saponins are basically classified as triterpenoids, steroids or steroid alkaloids, based on the structure of the aglycone, and monodesmosidic, bidesmosidic or tridesmosidic according to the number of sugar moieties attached to the aglycone. Hexoses (glucose, galactose), 6-dehydroxyhexoses (rhamnose, furanose), pentoses (xylose, arabinose) and uronic acids (glucuronic, galacturonic) are the most common sugar residues in the saponin molecules. The sugar moiety is linked to the aglycone through an ether or ester glycosidic linkage. Saponins have been known to cause substantial enhancement in immune responses, hence they have been used as delivery adjuvants in veterinary in vaccines. Many saponin-containing plants have been exploited for commercial saponin production. Although it has been reported that B. aegyptiaca contains saponins, there is no report of commercial saponin production from this plant.

Several studies on the B. aegyptiaca saponins have been reported (Liu and Nakanishi, 1982; Pettit et al., 1991; Hosny et al., 1992; Kamel, 1998; Kamel et al., 1995). Almost all parts of the B. aegyptiaca tree contain saponins, but the fruit mesocarp contains the highest extractable amount of saponins in comparison to other parts. Hosny et al., 1992, isolated and characterized the main saponin from the methanolic extract of the mesocarp of B. aegyptiaca, but attributed a wrong site of attachment of the rhamnosyl residue in the sugar chain of the molecule.

With increasing beneficial uses for saponins, identification of saponins in different plants has taken on greater significance in phytochemistry. Traditional methods for identifying saponins in plants are complicated and time-consuming because saponins are highly polar, thermally labile and structurally complex molecules. Therefore, there is an increasing demand for better methods for identifying and structurally characterizing saponins.

### SUMMARY OF THE INVENTION

Using liquid chromatography-electrospray ionization-mass spectrometry (LC-ESI-MS) technique, we succeeded in identifying and characterizing saponins found in B. aegyptiaca extracts.

Thus, in one aspect, the present invention relates to a saponin of the general formula I:

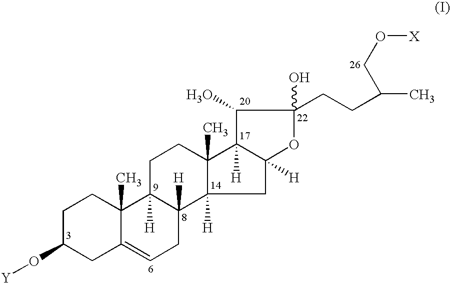

wherein

X is absent or is a glucose residue linked though its C1 position;

Y is absent or is a sugar chain selected from the group consisting of:

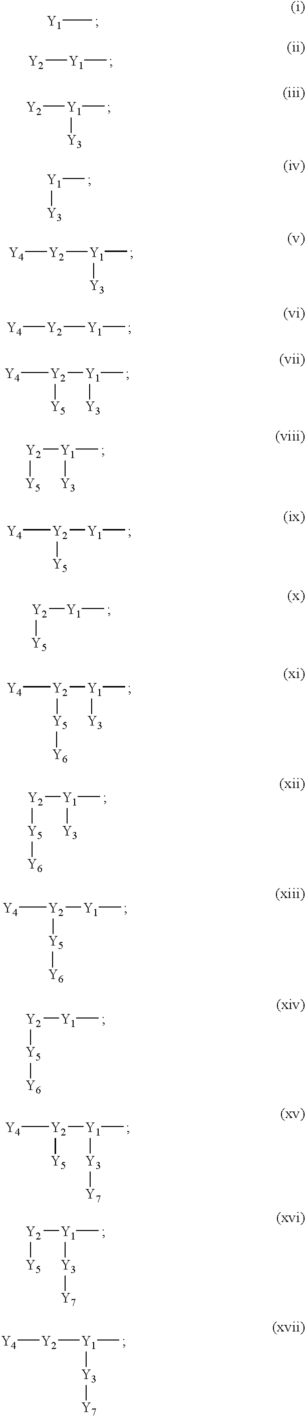

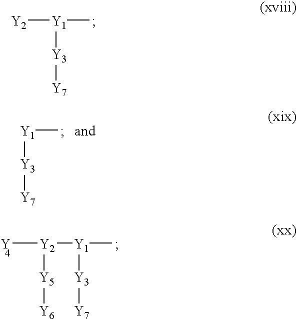

wherein

Y₁ is a glucose residue linked through its C1 position;

Y₂ is a glucose residue linked 1→4 to Y₁;

Y₃ is a rhamnose residue linked 1→2 to Y₁;

Y₄ is a xylose or a rhamnose residue linked 1→3 to Y₂;

Y₅ is a glucose residue linked 1→2 to Y₂;

Y₆ is a glucose residue linked 1→4 to Y₅, and

Y₇ is a glucose residue linked 1→4 to Y₃,

and hydrates and methylated derivatives thereof, but excluding the compound wherein X is a glucose residue and Y is the sugar residue (iii), and mixtures of said saponins of formula I.

It has further been found in accordance with the present invention that mixing saponins in aqueous solution, in a concentration that is above the critical micelle concentration (CMC), results in the formation of relatively high stable nanovesicles, which can encapsulate biological or chemical materials and protect them. Moreover, the interaction between saponins and biological membranes due to the high affinity of saponins to the sterols in the membranes makes these nanovesicles a good biodelivery system highly efficient to carry various biological and chemical materials through biological membranes.

Thus, in another aspect, the present invention relates to a stable preparation of saponin nanovesicles encapsulating an active material. Any saponin or plant extract rich in saponins can be used for this purpose such as Quillaja saponaria, B. aegyptiaca and Balanites roxburghii saponins and extracts.

It has also been found that plant extracts rich in saponins such as Quillaja saponaria and B. aegyptiaca accelerate the delivery of herbicides trough isolated cuticle membranes and can thus be used as foliage and root system penetrants for the delivery of agrochemicals.

Thus, in another aspect, the invention provides an agricultural composition for foliar application comprising an agrochemical and a saponin-rich plant extract. The agrochemical may be a nutrient, a plant growth regulator, or a pesticide.

It has further been found that adding saponin-rich plant extracts such as B. aegyptiaca extracts rich in saponins to irrigation water applied to the bases of cuttings and seedlings significantly increased the number of roots formed and their length and addition of said extracts to low quality saline irrigation water enhanced the germination of the seeds and the development of the germinated seedlings.

Thus, in a further aspect, the present invention relates to saponins such as B. aegyptiaca saponins as an adjuvant for irrigation water.

In one embodiment, the present invention provides a method of enhancing the rooting of cuttings comprising applying to the bases of the cuttings an aqueous solution of the B. aegyptiaca saponins, optionally together with the plant hormone indole-3-butyric acid (IBA).

In another embodiment, the present invention provides a method of enhancing the germination of crops seeds and the development of germinated seedlings irrigated with low quality saline water, comprising adding B. aegyptiaca saponins to the irrigation water.

### BRIEF DESCRIPTION OF THE FIGURES

FIG. 1 shows the LC-RI; MS; LS chromatogram of Balanites aegyptiaca fruit mesocarp.

FIG. 2 shows the time-course effect of Balanites mesocarp (ME), kernel (KE) and root (RE) saponins, in comparison to Quillaja saponaria extract saponin as well as to deionized distilled water (negative control) and the nonionic surfactant Triton X-100 (positive control), on the penetration rate of ¹⁴C labeled 2,4-D across the Citrus grandis leaf cuticle membrane (CMs) at 30° C. and 30% relative humidity. Each treatment contained the same concentration of 2,4-D together with 1% (w/v) solution of either Triton, ME, RE, KE or QE as an adjuvant. QE, ME, KE, RE and DDW refer to Quillaja saponaria extract saponin, Balanites aegyptiaca mesocarp extract saponin, B. aegyptiaca kernel extract saponin, B. aegyptiaca root extract saponin and deionized distilled water, respectively. The DDW contained only 2,4-D solution as a control. Each value is the mean of 60 CM±SE.

FIG. 3 shows the time-course effect of various concentrations of Balanites aegyptiaca mesocarp saponin (ME) on the penetration rate of ¹⁴C labeled 2,4-D across the Citrus grandis leaf cuticle membrane (CMs) at 30° C. and 30% relative humidity. Each treatment contained the same concentration of 2,4-D together with the amount of ME as an adjuvant as listed on the graph. The control contained no any adjuvant and just 2,4-D and DDW. Each value is the mean of 60 CMs±SE.

FIGS. 4A-4D show the transmission electron microscope (TEM) characterization of the nanovesicles present in the different saponin solutions (0.50%), with uranyl acetate background as negative staining in 25K magnification. (A) Quillaja saponaria extract saponin (QE); (B) B. aegyptiaca fruit mesocarp extract saponin (ME); (C) B. aegyptiaca kernel extract saponin (KE); and (D) B. aegyptiaca root extract saponin (RE).

FIGS. 5A-5B show the SOD (A) and GSH-Px (B) isozyme patterns on native gels as a result of treatment with B. aegyptiaca mesocarp (ME) and root (RE) saponins, as well as with Q. saponaria bark extracted saponin (SS). Each lane was loaded with 25 μg of total protein from cuttings treated with water (C) or with B. aegyptiaca mesocarp extracted saponin (ME), B. aegyptiaca root extracted saponin (RE) and Q. saponaria bark extracted saponin (SS), each at a concentration of 500 ppm for 6 h or 24 h. Arrows indicate different enzyme isoforms.

FIG. 6 shows the inhibition effect of B. aegyptiaca purified main saponins from mesocarp (ME), root (RE) and kernel (KE), at a concentration of 1% w/v, on Escherichia coli growth.

### DETAILED DESCRIPTION OF THE INVENTION

The present invention relates, in one aspect, to the saponins of the general formula I. These saponins are extracted from the Balanites aegyptiaca fruits (mesocarp or kernel), roots, kernel cake or oil.

The Balanites aegyptiaca extracts are obtained by methods known in the art. The various saponins of general formula I herein are then isolated from the methanolic extract, analyzed by LC-EIS-MS or LC-EIS-MS/MS, and confirmed by 800 Mhz NMR.

The term “Balanites aegyptiaca saponins” as used herein denotes a saponin of the general formula I herein, a mixture thereof, or a Balanites aegyptiaca extract rich in said saponins. The extract may be the mesocarp, root, kernel cake or oil extract.

In one aspect of the present invention, nanovesicles are produced from plant saponins by mixing them with water in a concentration that is above the critical micelle concentration (CMC). The CMC is a measure of the concentration of a solution component that represents a critical value above which increasing concentration of that component forces formation of micelles. For the preparation of the nanovesicles, any plant saponins are encompassed by the invention such as, but not limited to, Quillaja saponaria and Balanites aegyptiaca saponins. For this purpose, isolated saponins or plant extracts rich in the saponins can be used. In one preferred embodiment, B. aegyptiaca saponins extract is used although also isolated saponins of formula I or mixtures thereof can also be used.

The nanovesicles can encapsulate an active material dissolved in the water forming stable preparations. Thus, in another aspect, the present invention provides stable preparations of saponin nanovesicles encapsulating an active material.

The active material is either organic or inorganic and may be, for example, a drug, a toxin, a pesticide, a vitamin, a hormone, a plant growth substance, a mineral, a nutrient, a nucleic acid, an aroma and flavor compound, a flavonoid, a colloid, or a mixture thereof.

In one embodiment of the invention, the active material is a pesticide such as, without being limited to, an herbicide, e.g., 2,4-dichloro-phenoxyacetic acid (2,4-D) and an insecticide.

In one preferred embodiment, the active material is a toxin from a microorganism useful as a biopesticide including, but not limited to, Bacillus thuringiensis israelensis (known as Bti) toxin. Bti toxin is a known bioinsecticide highly specific to mosquito and blackfly larvae, with negligible effects on non-target invertebrate or vertebrate organisms. Therefore, it is the main approved treatment for larvae control worldwide and currently represents about 1% of the total ‘agrochemical’ world market. However, the Bti toxin is sensitive to UV irradiation (sunlight) and therefore has a toxic action of short duration (only several hours) after application. The Bti toxin also has a short period of activity in flooded areas due to the high organic matter content in swamps and rice paddies, that cause the Bti toxin to sink to the low water column that is too far away from the larvae growing water surface. Encapsulation of Bti toxin in the Balanites saponin nanovesicles protects the toxin from inactivation by UV irradiation and may significantly extend its larvicidal activity. Moreover, the amphipathic nature of the saponin prevents the Bti toxin from fast sinking to the lower column thus maintaining the product in the upper column at the larvae-developing zone.

Thus, in one preferred embodiment, the invention provides a pesticidal composition comprising a stable preparation of saponin nanovesicles comprising a core of Bti toxin encapsulated within the saponin nanovesicles. In one preferred embodiment, the nanovesicles are made from Balanites aegyptiaca saponins. The invention further provides a method of controlling the growth of mosquito larvae comprising dispersing said pesticidal composition over agricultural areas affected by said larvae.

In another embodiment, the active material encapsulated within the saponin nanovesicles is a lipophilic or hydrophilic vitamin. The vitamins that can be encapsulated according to the invention include the vitamins A, B, C, D, E, F, K, P, or mixtures thereof.

In one embodiment, the vitamin is vitamin A, either in its free form as retinol or in its ester form as retinyl palmitate. Retinol is an anti-oxidant vitamin used as nutritional factor and also as an active ingredient of topical/dental products and can be used for topical treatment of Ichthyosis vulgaris (an inherited skin disorder characterized by cornification of the skin) and common acne, and in anti-aging and rejuvenation formulations. However, retinol (an unsaturated alcohol) is a small and unstable molecule and undergoes chemical degradation/oxidation due to its high potential for chemical reactions with other molecules and should be stabilized before using it as an active ingredient in compositions. In order to enjoy the beneficial effects of retinol and meet the shelf-life needed for topical/dental compositions, this active material should be protected from oxidation. Encapsulation of retinol by the saponin nanovesicles of the invention may allow its use in various applications including, without limiting, dental products, anti-aging products (creams, lotions, serums and masks), skin regeneration formulations, nourishing and moisturizing creams and anti-acne products.

In another embodiment, the vitamin is vitamin C (ascorbic acid), used in recent years as an active ingredient of cosmetics or a derivative thereof such as ascorbyl palmitate and magnesium ascorbyl phosphate. Due to its antioxidant properties, it is considered to confer both antioxidant and photoprotection to skin against free radical attack and UV ray damage. However, vitamin C is easily oxidized and, upon storage, exposure to light, oxygen, moisture and/or high temperature, undergoes rapid degradation. It is unstable in aqueous solution, even under neutral pH and at room temperature. The encapsulation of vitamin C according to the present invention permits its use as active ingredient in cosmetic composition for use as moisturizing cream, anti-aging cream, anti-wrinkle cream, sunscreen cream, and for stimulating collagen production.

In another embodiment, the vitamin is vitamin E, preferably as α-tocopherol. Tocopherols (vitamin E) are well-known for their antioxidant properties making vitamin E one of the most widely consumed vitamins. However, vitamin E in its ester form (e.g., tocopherol acetate) is only effective as antioxidant to the formulation, but not to the skin. To be effective as antioxidant to the skin, α-tocopherol has to be used, but it is inherently unstable. The nanovesicles of the invention preferably contain stable 25±1% α-tocopherol, and can be used in various types of cosmetic formulations such as sunscreen products, shampoos, conditioners, hair gels, liquid make-up and make-up tissue remover, and release about 95-97% of vitamin E directly onto the skin/scalp upon application.

In a further embodiment, the vitamin is vitamin F, a mixture of unsaturated fatty acids essential for skin health and functionality, also known as Essential Fatty Acids (EFA; linoleic acid and alpha-linolenic acid). Vitamin F oxidizes rapidly when incorporated in cosmetic formulation. The encapsulation in the nanovesicles according to the invention offers a stable, active and odorless system of vitamin F suitable for incorporation into moisturizing creams, anti-aging agents and anti-dryness serums.

In another embodiment, the vitamin is rutin (quercetin-3-rutinoside or vitamin P1), one of the most active natural flavanoids, highly effective as an antioxidant and free radical scavenger and in the treatment of cellulite due to its ability to control cross-linking of collagen synthesis. Rutin is widely applied in dermatological and cosmetic products due to its beneficial effects on the appearance of healthy skin and is well known for its potent antioxidant and anti-inflammatory properties and ability to strengthen and modulate the permeability of the walls of the blood vessels including capillaries. However, when incorporated into cosmetic formulations in its non-encapsulated form, rutin tends to react with other ingredients and oxidizes quickly, resulting in change of the original color of the formulation and loss of its original biological activity. In order to maintain its potent biological activity and prevent its oxidation in cosmetic formulations, rutin should be stabilized. The rutin nanovesicles of the present invention may be used for development specifically of preparations for topical application in order to stabilize the rutin.

Thus, the present invention further provides stable preparations of saponin nanovesicles encapsulating a vitamin and dermatological or cosmetic composition comprising at least one such preparation. In a preferred embodiment, the nanovesicles are made from Balanites aegyptiaca saponins and the vitamin is vitamin C.

Besides the encapsulation of Bti for use as a biological insecticide, the invention encompasses also other uses of the saponin extracts in the agriculture.

Foliar application is an effective but often inefficient method for applying agrochemicals to the crop plants. However, in intensive agriculture, the practice of foliar application of nutrients, growth regulators, pesticides and herbicides is increasing. The efficacy of these foliage-applied agrochemicals depends on the amount of active ingredients penetrating across the plant cuticle layer that covers all the external surface of plants, including leaves of higher plants, and is the main barrier to the penetration of foliar-applied agrochemicals. The use of agricultural adjuvants and/or surfactants has become common practice in foliar application in order to enhance the delivery of the agromaterials to the inner tissue of the plant through the cuticular layer.

It has been found in accordance with the present invention that plant extracts rich in saponins such as Quillaja saponaria and B. aegyptiaca accelerate the delivery of herbicides through isolated cuticle membranes and can thus be used as foliar penetrants for the delivery of agrochemicals.

Thus, in another aspect, the invention provides an agricultural composition for foliar application comprising an agrochemical and a saponin-rich plant extract. The agrochemical may be a nutrient, a plant growth regulator, or a pesticide. In one preferred embodiment, the saponin-rich plant extract is the B. aegyptiaca saponin extract and the pesticide is an herbicide, preferably 2,4-D.

It has further been found that adding B. aegyptiaca extracts rich in saponins to irrigation water applied to the bases of cuttings significantly increased the number of roots formed and their length and addition of said extracts to low quality saline irrigation water enhanced the germination of the seeds and the development of the germinated seedlings.

Thus, in a further aspect, the present invention relates to saponins, particularly B. aegyptiaca saponins, as an adjuvant for irrigation water.

In one embodiment, the present invention provides a method of enhancing the rooting of cuttings comprising applying to the bases of the cuttings an aqueous solution of the B. aegyptiaca saponins, optionally together with the plant hormone indole-3-butyric acid (IBA).

In another embodiment, the present invention provides a method of enhancing the germination of crops seeds and the development of germinated seedlings irrigated with low quality saline water, comprising adding B. aegyptiaca saponins to the irrigation water.

The use of Q. saponaria has already been exploited for commercial uses, but the use of Balanites is still very negligible. Since Balanites is highly adopted in most of the arid lands where other crops can hardly be grown, the production of the environmentally friendly B. aegyptiaca saponins could be cheap and economically attractive. The antifungal and antimicrobial properties of these saponins contribute to an integrated pest management concept.

The present invention thus provides an integrated pest management concept based on the ability of saponins in general, and of B. aegyptiaca saponins in particular, to increase the uptake of water, including saline water, to protect agrobiological and agrochemical materials and to deliver said materials through irrigation systems, and to afford antifungal and antimicrobial protection to agricultural areas.

It has also been found that B. aegyptiaca saponins have a natural biocide activity in three major biotic fields closely related to aqueous media, in particular, in larvae control, fungi control and bacteria control. Thus, in another aspect, the present invention relates to a method of inhibiting the growth of mycelial colonies affecting plants, comprising dispersing over agricultural areas an aqueous solution of B. aegyptiaca saponins.

Mixing plant saponins with a lipophilic medium in a concentration that is above the critical micelle concentration (CMC) forms inverse micelles that can complxate various materials such as water residue, toxic ions and biomaterial. In petroleum fuel and/or biofuel, such inverse micelles may be used as an adjuvant for capturing water residue and toxic ions residue, thereby extending the engine system life, enhancing its performance and reducing sulfur and other related toxic elements emission. For the preparation of the inverse micelles, any plant saponins are encompassed by the invention such as, but not limited to, Quillaja saponaria and B. aegyptiaca saponins. For this purpose, isolated saponins or plant extracts rich in the saponins can be used. In one preferred embodiment, B. aegyptiaca saponins extract is used although also isolated saponins of formula I or mixtures thereof can also be used.

Thus, the present invention further provides a method of extending life of a petroleum fuel or biofuel based engine, enhancing the performance of said engine and reducing the emission of toxic elements from said engine, comprising adding saponins or saponin-rich plant extract such as Quillaja saponaria or B. aegyptiaca saponins or extract to the petroleum fuel or biofuel consumed by said engine.

The invention will now be illustrated by the following non-limiting Examples.

### EXAMPLES

### Example 1

### Selection of Balanites aegyptiaca Superior Genotype Developing in Semi-Arid Area Using Low Quality Saline and Sewage Water

(i) B. aegyptiaca Germplasm Collection.

Matured fruits were collected from various populations distributed in the whole geographic range of B. aegyptiaca worldwide distribution areas in the year 1996. B. aegyptiaca seeds were produced from fruits collected from two sites in Djibouti and Eritrea areas; two sites in the Dakar area, Senegal; two sites in the Bamako area, Mali; and two areas in Jodhpur, India. B. aegyptiaca fruits were collected from Israeli genotype trees in Ein Gedi, Eilat, Samar, Sapir, Sde Taiman and Kfar Rupin (considered as the northernmost limit of Balanites distribution—35°25′N). The Balanites trees of Ein Gedi and the Samar areas seemed wild vegetation whereas the Eilat, Sapir, Sde Taiman and Kfar Rupin trees were probably based on a local Israeli collection from some years ago. The Balanites plant species were identified taxonomically by Prof. Uzi Plitman from the herbarium of the Hebrew University of Jerusalem. Voucher specimen (76816) was deposited in the herbarium of the Hebrew University of Jerusalem for the Mali specimen, herein designated B3.

Collected fruits were soaked in water and, after 48 hours, the outer parts of the fruits, epicarp (outer cover) and mesocarp (pulp), were removed. The nuts were cleaned, placed in boxes containing one-inch layer of vermiculite and kept moistened by hand irrigation. The boxes were placed in a greenhouse. Nuts started to germinate after 25 days and were then transferred to a polyethene bag containing soil, vermiculite/perlite (1/1 w/w) and continuously irrigated.

One-year-old seedlings were planted in 1997 in introduction orchards established at Kibutz Samar at the Arava Rift Valley (South Negev, Israel). This area is characterized by summer high day temperatures, low humidity, relatively low night temperatures and relatively warm winter climate. Eilat partially purified sewage water, characterized by average high EC of 5 dS·m⁻¹, was supplied to the seedlings since planting. The seedlings were planted in several rows with 4 m between plants and 6 m between the rows. The experimental design of the plot was based on three blocks and three replicates of each genotype. Water and soil analysis was carried out routinely several times a year.

(ii) Characterization of B. aegyptiaca Vegetative Organs Development.

At the end of the summer of every year, the development of foliage of each genotype in the plot was estimated based on the following height scale: + (main shoots)<2 m height; ++ (main shoots)˜3 m height; +++ (main shoots)>4 m height.

The development of the root system of the collected Balanites genotypes was initially studied using controlled grown seedlings in Beer Sheva on sandy soil (similar to Samar plot soil) and irrigated with saline water (EC of 5 dS·m⁻¹). This controlled trial demonstrated the development of unique double root system based on upper well-branched system close to the ground surface and a second deeper (several meter depth) and branched root system. This system provides the Balanites trees an increased capacity of water uptake in arid areas. The present study clearly showed that the high saline irrigation water did not damage the plants.

Following this controlled trial, the development of the root system of each genotype was estimated in Samar plot conditions at the end of the summer of the fourth year. A mechanized router machine was operated at a distance of 6 m from the border row of trees in depth of about 2 m. The router enabled to observe the development of the upper root system of trees of all the genotypes. Estimation of the development and distribution of the roots from the trunk of each tree was carried out by the following scale: a (roots found)<4 m (radius from the trunk); aa (roots found)˜6 m (radius from the trunk); aaa (roots found)>8 m (radius from the trunk). Estimation of the depth of the roots of each genotype was done after digging to a depth of 5 m and testing soil samples. The following scale was used: y (roots found)<2 m depth; yy (roots found)˜5 m depth; yyy (roots found)>5 m depth.

All the local and the worldwide samples of Balanites collected germplasm were tested in a collection plot located in the south Negev, characterized by semi-arid conditions. The plants were irrigated with low quality wastewater partially purified and containing high level of salinity. Following an intensive foliage and root system study, the Balanites genotype B3 was selected as a superior genotype among the tested genotypes (Table 1). B3 genotype developed the best foliage and root system in comparison to all the other genotypes. Typically to Balanites, all the genotypes produced a double root system that enabled an efficient water uptake from the soil in various depths. B3 genotype produced the most developed upper and lower root systems and its roots reached the deepest depth amongst all the tested Balanites genotypes.

(iii) Characterization and Yield of B. aegyptiaca Fruits.

Mature ripen abscised fruits were collected from the ground under each tree. Fruits of each tree were weighed and measured for its length and diameter individually. The outer cover of the fruit and pulp were carefully detached from the fruit and weighed. The fresh nuts were weighed, broken manually and kernels were taken out and weighed. The mesocarp (pulp) of the fruits was kept in fridge for further analyses. As shown in Table 2, the average fruit weight pulp and kernel percentage were not the highest among the other genotypes, however, the productivity of B3 was significantly increased in comparison to all the other tested Balanites genotypes.

TABLE 1
Balanites aegyptiaca selected genotypes foliage and root systems
response to irrigation with partially purified saline sewage
(EC 5 dS · m⁻¹) in Samar semi-arid area

Development*

Root Radius

| | Foliage | Upper | Lower | Root |
|---|---|---|---|---|
| Genotype | Height | system | system | depth |
| Samar | +++ | aaa | aa | yy |
| Ein Gedi | ++ | aa | aa | yy |
| Sapir | + | aa | a | yyy |
| B3 | +++ | aaa | aaa | yyy |
| B5 | ++ | aaa | aa | yyy |

*Development estimations were based on at least three replicates for each genotype.

TABLE 2
Balanites aegyptiaca selected genotypes yield in response to
irrigation with partially purified saline sewage (EC 5 dS · m⁻¹)
in Samar semi-arid area

| | Development* | | | |
|---|---|---|---|---|
| | Yield | Fruit Fresh | | |
| Genotype | (Kg/Tree) | wt (gr) | Pulp (%) | Kernel (%) |
| Samar | 29 ± 3.2 | 5.05 ± 0.18 | 33.0 | 9.0 |
| Ein Gedi | 18 ± 4.6 | 6.72 ± 0.19 | 26.6 | 8.5 |
| Sapir | 12 ± 5.2 | 5.90 ± 0.13 | 26.4 | 8.1 |
| B3 | 35 ± 4.1 | 6.06 ± 0.24 | 24.4 | 8.4 |
| B4 | 26 ± 3.7 | 5.25 ± 0.12 | 21.8 | 10.8 |
| B5 | 23 ± 6.0 | 2.46 ± 0.10 | 20.3 | 12.1 |

*Values are the mean of at least three trees for yield and of 25 fruits for fresh weight ± SE.

(iv) B. aegyptiaca Oil Characterization and Saponin Determination.

The oil from the kernel of each genotype was extracted with hexane and oil percentage was determined. Oil quality parameters such as fatty acid profile were determined by standard oil analysis protocols (CODEX STAN 33).

As shown in Table 3, the oil percentage of the B3 genotype was in the average range of the tested Balanites genotypes. The fatty acid profile of all the Balanites genotypes oil fits well the food and industry common standards.

The saponin level was determined in the mesocarp and the roots of each genotype using the spectrophotometric method as described by Uematsu et al. (2000) and Baccou et al. (1977). As shown in Table 4, B3 genotype accumulated the highest saponin level both in the mesocarp (pulp) and in the root in comparison to all the other tested Balanites genotypes.

Taking all these parameters into consideration, B3 Balanites genotype was selected as a superior genotype in terms of extremely well development in semi-arid conditions and under irrigation with low quality water containing high saline level. Under these conditions, this selected genotype produces intensive vegetative organs that may contribute, when distributed in semi-arid areas, in large scale to the global CO₂ balance. The extremely efficient root system developed by the B3 genotype may be useful for industrial wastewater uptake in semi-arid areas and may assist in the reclamation of contaminated areas. The selected B3 Balanites genotype yields large amounts of fresh edible Balanites fruits, producing high amounts of food grade oil. The additional important raw material produced by the B3 Balanites genotype, the saponins, are highly required by a wide range of advanced industries such as food, pharmaceutical, agriculture, environment, etc.

TABLE 3
Balanites aegyptiaca selected genotypes oil percentage and fatty
acid profile in response to irrigation using partially purified saline
sewage (EC 5 dS · m⁻¹) in Samar semi-arid area

| | | Fatty Acid Profile* | | | | | |
|---|---|---|---|---|---|---|---|
| Genotype | Oil (%) | C16:0 | C18:0 | C18:1 | C18:2 | C18:3 | Others |
| Samar | 45.1 ± 1.10 | 12.79 | 12.16 | 34.95 | 38.35 | 1.16 | 0.59 |
| Ein Gedi | 46.7 ± 0.59 | 13.70 | 11.04 | 43.76 | 31.50 | 0.00 | 0.00 |
| Sapir | 39.0 ± 1.35 | 13.71 | 10.55 | 23.58 | 51.61 | 0.47 | 0.09 |
| Eilat | 42.1 ± 2.00 | 14.02 | 11.10 | 28.23 | 46.20 | 0.01 | 0.44 |
| B3 | 44.1 ± 1.02 | 14.60 | 10.25 | 31.82 | 43.34 | 0.00 | 0.00 |
| B4 | 43.2 ± 1.10 | 12.97 | 11.85 | 30.77 | 44.00 | 0.42 | 0.00 |
| B5 | 46.0 ± 1.40 | 16.03 | 10.54 | 27.84 | 44.68 | 0.35 | 0.57 |

*Values are the mean of at least 4 samples ± SE

TABLE 4
Balanites aegyptiaca selected genotypes saponin (disogenin)
production in response to irrigation using partially purified
saline sewage (EC 5 dS · m⁻¹) in Samar semi-arid area

Saponin*

| Genotype | Mesocarp | Root |
|---|---|---|
| Samar | 11.7 ± 0.4 | 13.1 ± 0.6 |
| Ein Gedi | 10.4 ± 0.8 | 11.8 ± 0.4 |
| Sapir | 9.3 ± 0.4 | 10.5 ± 0.4 |
| Eilat | 9.6 ± 0.6 | 10.9 ± 0.6 |
| B3 | 12.6 ± 0.4 | 13.7 ± 0.4 |
| B4 | 10.2 ± 0.6 | 12.6 ± 0.6 |
| B5 | 11.3 ± 0.6 | 12.6 ± 0.6 |

*Values are the mean of at least 4 samples ± SE

### Example 2

### Extraction of Balanites aegyptiaca Saponins (Small Scale)

(i) Saponin Extraction from the Balanites Fruits.

Fully ripened fruits of B. aegyptiaca were collected from Balanites aegyptiaca grown at Kibutz Samar. The epicarp (outer cover) was removed by hand and the mesocarp (pulp) was manually extracted with a knife. The mesocarps were first freeze-dried with a lyophilizer (Christ Alpha 1-4, Germany) and then stored in an electric desiccator (Sanplatec Corp., Israel) for further use.

For the extraction, the freeze-dried mesocarp was pulverized, combined with methanol (1:10) and shaken continuously overnight in a high-speed electric shaker (Tuttnauer, Jerusalem, Israel) followed by centrifugation (3500 rpm, 18 min, 20° C.), and supernatants were collected. The residue was further extracted twice using vortex and centrifugation. After three successive extractions, the supernatant was clear. All the supernatants were combined and the methanol was evaporated off in a rotary evaporator (Heta-Holten A/S, Denmark) under reduced temperature (below 40° C.). The residue was dissolved in water (1:2 w/v), the extract was then defatted three times using a 1:2 ratio of n-hexane. The water was then removed from the defatted extract with a lyophilizer. The semicrystalline dried saponin extract was designated ME (methanol extract).

(ii) Saponin Extraction from the Balanites Roots.

Fresh roots of matured plants of B. aegyptiaca grown in Kibutz Samar were collected, chopped into small pieces and oven dried at below 50° C. The dried pieces were then powdered by a coffee grinder. The powder was first defatted by n-hexane and then extracted with MeOH. In brief, 3 g of root powder were taken in a plastic tube (50 ml capacity, centrifugeable grade) and 30 ml MeOH were added. The tubes were put over night in an electric shaker (Tuttnauer Ltd, Jerusalem, Israel) in high speed and then were centrifuged (3500 rpm, 18 min, 20° C.), and supernatants were collected. The precipitate was further extracted twice using vortex and centrifugation. After three successive extractions, the supernatant was clear. All the supernatants were combined and the MeOH was evaporated using a rotary evaporator, thus obtaining a yellowish crystal powder of crude saponins (˜12.2% of dry root weight), herein named RE (root extract). The RE was dissolved in methanol:water (70:30) in 1 to 100 ratio, filtered by 0.45 μm, and used for the LC-EIS-MS and LC-EIS-MS/MS analysis.

(iii) Saponin Extraction from the Balanites Kernels and Oil.

Two kg well ripen fresh B. aegyptiaca fruits were collected from the B. aegyptiaca grown in Kibutz Samar. The epicarp and mesocarp of the fruits were removed and the nuts were washed by tap water and oven dried at below 70° C. for 72 h. Decortication of the nuts (by hand) release the kernel (200 g, ˜10% of the total fresh weight). The kernel and endocarp (stone) ratio was 30:70 by weight. The kernels were coarsely ground and kept in refrigerator until further work.

For oil extraction, 3 g of course grounded kernels were taken in and 3 extractions were carried out using n-hexane. In brief, 3 g of kernel powder were taken in a plastic tube (50 ml capacity, centrifugeable grade) and 30 ml n-hexane were added. The tubes were put over night in an electric shaker (Tuttnauer Ltd, Israel) in high speed and then were centrifuged (3500 rpm, IS min, 20° C.), and supernatants were collected. The residue was further extracted twice using vortex and centrifugation. After three successive extractions, the supernatant was clear. All the supernatants were combined and the n-hexane was evaporated using a rotary evaporator. The oil was collected. A range of 45-50% oil by w/w was obtained.

The cake (kernel left over) was kept under the hood overnight in order to dry all the hexane. In the next day, 30 ml MeOH was added to each tube and kept over the shaker overnight followed by centrifugation. The second and third extractions by methanol were carried out as with hexane. At the end, all the supernatants of the methanol extract were pooled and the methanol was evaporated using a rotary evaporator, thus obtaining a yellowish crystal powder of crude saponins (˜12.2% of kernel weight), herein named KE (kernel extract). Ten tubes were used for 30 g of kernel powder. This powder was kept in refrigerator and used for further analysis.

(ii) Total Saponins in Different Tissues of the B. aegyptiaca.

Sapogenin amounts in each extract were determined by measuring absorbance at 430 nm, based on the color reaction with anisaldehyde, sulfuric acid and ethyl acetate as described by Uematsu et al. (2000) and Baccou et al. (1977) with some modification. Total saponins were calculated based on the mass of diosgenin (steroid aglycone of the major Balanites saponins, MW 414) and major saponins present in the extracts (Uematsu et al., 2000). For the saponin calculation, the mass of the major saponins in mesocarp extract (ME) was taken as MW 1064, MW 1210 for root extract (RE) and kernel extract (KE) as compared to the MW 414 for diosgenin (see Example 4 hereinafter). As shown in Table 5, B. aegyptiaca tissues (mesocarp, kernel and root) produce extremely high levels of saponins. For example, tissues of other plant species such as mung bean (Vigna radiate) or olive (Olea europea) were tested as well and found to produce less than 1% of sapogenin.

TABLE 5
Total saponins in different tissues of the Balanites aegyptiaca

| Balanites tissue | Sapogenin, % | Saponins, % |
|---|---|---|
| Mesocarp (ME) | 12.23 | 32.41 |
| Kernel (KE) | 14.32 | 37.95 |
| Root (RE) | 13.18 | 34.93 |

### Example 3

### Pilot Scale Extraction of Balanites aegyptiaca Mesocarp Saponins

Batches (20 kg) of whole Balanites fruits were placed in a 150 kg rotating pan containing about 80 liters of water. The pan was rotated for several hours until the mesocarp was dissolved and washed well from the seeds. The seeds were separated from the aqueous solution containing the mesocarp glycosides and the solution was filtered to remove solid residues. A sample of the aqueous solution was defatted with light petroleum ether (b.p. 60-80° C.) and then dried by lyophilization. The dried crystallized glycoside batch was dissolved in methanol and loaded on a C18 Sep-Pack (Waters) large-scale column. The column was first washed well with water to elute the free sugars and then washed with methanol to elute the partially purified glycoside conjugated saponins.

### Example 4

### Chemical Characterization of Balanites aegyptiaca Saponins

The liquid chromatography-mass spectrometer (LC-MS/MS) technique has already been used to detect and identify saponins from plant extracts (Liu et al., 2003; Cui et al., 2000). This method has proven to be efficient and has become the method of choice. We use herein the liquid chromatography-electrospray ionization-mass spectrometry (LC-ESI-MSⁿ) technique.

(i) Chemical Characterization of Saponins from Balanites mesocarp.

The saponins of the methanol-extracted B. aegyptiaca mesocarps were separated by high-performance liquid chromatography-refractive index (HPLC-RI) to detect the whole spectrum of its major saponins, followed by electrospray ionization-mass spectrometry (ESI-MS) combined with multistage mass spectrometry (MSⁿ) to identify the major steroidal saponins. Detection and identification were based on the ion mass and fragment ion mass of each separated peak. A purified authenticated standard was also used to validate the system.

Liquid chromotography. A Waters (Milford, Mass., USA) 2690 HPLC system equipped with an auto sampler, a Luna 5 u 100 A C18 analytical column (250×2 mm, 5 μm), an HPLC pump and an Agilent (Palo Alto, Calif., USA) G1314A RI detector, were operated at room temperature. The mobile phase was 70:30 MeOH:H₂O at a flow rate of 0.2 ml/min. The injection volume was 10 μl. The ME was eluted in methanol in solid phase extraction (SPE) cartridges (C18, 35 ml, 10 g), after discarding the first elution in water before injection. The evaporative light scattering (ELS) detector was a Sedex 75 (Sedere, France). Luna C18 analytical column (250×2 mm, 5 μm particle size) (Phenomenex, USA), equipped with a guard, was used for the separations, with the same isocratic mobile phase of 70:30 MeOH:H₂O. A 100 μl sample was injected at a flow rate adjusted to 0.3 ml/min. The ELS detector separation was performed at ambient temperature. The ELS detector probe temperature was set at 50° C., the gain was set at 9 and the nitrogen nebulizer gas was adjusted to 2.0 bar.

Mass spectrometry. All the experiments were performed with a single ion-trap mass spectrometer (Esquire 3000 Plus, Bruker Daltonik) equipped with an ESI interface as the ion source for MS analyses. The electrospray voltage was set at 4.5 kV. The temperature of the ion source capillary was 300° C. Negative ion mode was used for all experiments other than the standards, in which case both negative and positive ion modes were used. Mass spectrometer conditions were optimized to achieve maximum sensitivity. Full scan spectra from m/z 25 to 2000 in the negative ion mode were obtained (scan time 1 sec). The ion trap was set for acquisition in automatic gain control mode with an accumulation time of 159 μsec. For MSⁿ analyses, the isolation width for [M-H]⁻ molecular ions was 3 m/z units; for MSⁿ experiments, fragmentation was induced using activation amplitudes of 0.95 to 1.12 V. Conformation of the ESI-MS peak identification was done by using a purified standard sample of B. aegyptiaca saponin (MW 1064). The standard sample purification and elucidation were carried out by the Royal Danish School of Pharmacy, Denmark, using 800 MHz nuclear magnetic resonance (NMR).

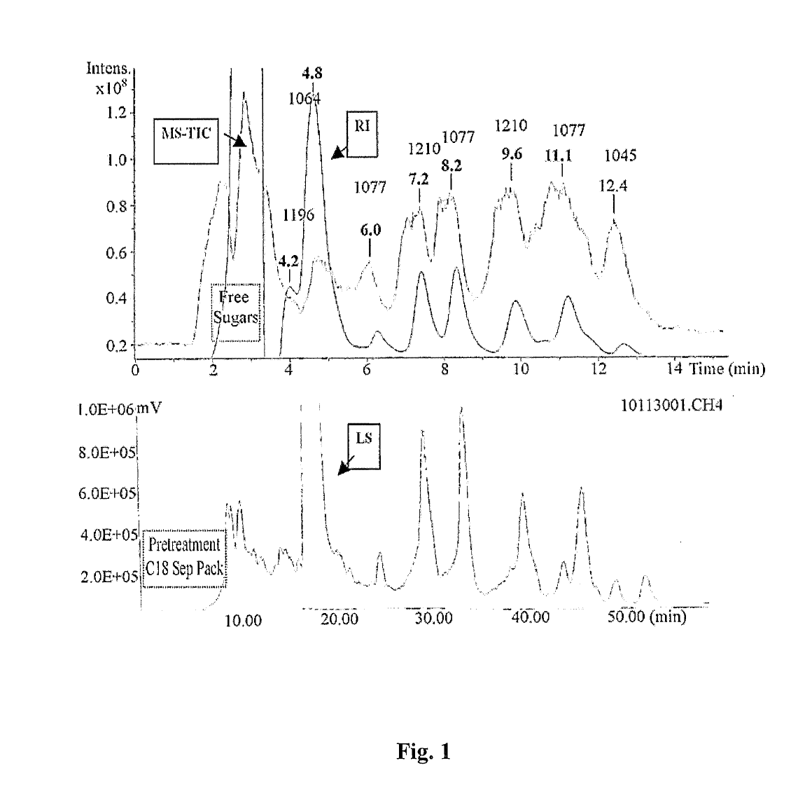

With the HPLC analysis, eight peaks were detected in the methanol extract of the mesocarp of B. aegyptiaca fruits, with one major peak. Mass analysis of each peak revealed 5 saponins corresponding to molecular masses of 1196, 1064, 1078, 1210 and 1046 Da (FIG. 1). These saponins are steroidal saponins with diosgenin as aglycone (MW 414, also found in a hydrated form of MW 432) and glucose (Gluc), rhamnose (Rham) and xylose (Xyl) as sugar units attached to it.

The main mesocarp extract saponin (about 40% of the total saponins) of molecular mass 1064, is the compound 26-(O-β-D-glucopyranosyl)-3β,22,26-trihydroxyfurost-5-ene 3-O-β-D-glucopyranosyl-(1→4)-[α-L-rhamnopyranosyl-(1→2)]-β-D-glucopyranoside, composed of the aglycone diosgenin, a Gluc unit at position C26 and a Gluc-Gluc; Rham chain at position C3. This saponin was first reported by Hosny et as (1992), but the formula reported was wrong (the use of acetylation-induced shifts yielded erroneous information about the attachment of the Rham residue to C-2″ instead of to C-2′). The correct formula of the MW 1064 saponin glycoside was demonstrated by using a 800 MHz NMR system, as reported by the present inventor and colleagues in a poster presented on a presentation day of the Royal Danish School of Pharmacology, in the year 2002.

The other minor saponins of the methanolic mesocarp extract are also found structurally very similar to the major saponin of molecular mass 1064, with the addition of one xylose (molecular mass 1196), one rhamnose (molecular mass 1210) or additional methyl group (molecular mass 1078) at C22 due to use of methanol as a solvent. A further saponin (molecular mass 1210) is similar to compound 1196 but with an additional methyl group at C22. The formation of methoxy derivatives by addition of an —OCH₃ group in place of an —OH group during extraction with methanol as found with compounds of molecular mass 1078 and 1210 has been reported in earlier studies of furostanol saponins (Hostettman and Marston, 1995). Two isomers of the compounds of molecular mass 1078 and 1210 were identified based on their different retention times from the C18 column used for LC (Table 6). The presence of isomeric saponins in plant extracts has been previously reported (Cui et al., 2000).

TABLE 6
Major HPLC-RI peaks of methanol-extracted B. aegyptiaca mesocarp
with m/z values obtained from ESI-MS

| | Retention | | |
|---|---|---|---|
| Peaks | time | m/z [M − H]⁻ | Area (%)* |
| 1 | 4.2 | 1196 | 2.7 |
| 2 | 4.8 | 1064 | 37.5 |
| 3 | 6 | 1077 | 1.9 |
| 4 | 7.2 | 1209 | 14.3 |
| 5 | 8.2 | 1077 | 11.2 |
| 6 | 9.6 | 1209 | 9.3 |
| 7 | 11.1 | 1077 | 8.6 |
| 8 | 12.4 | 1045 | 3.1 |

*Drawn from the m/z and MSⁿ spectral value of each peak with literature study

(ii) Chemical Characterization of Saponins from Balanites Kernel Cake.

A methanolic extract of B. aegyptiaca kernel cake of Example 2(iii) above was analyzed for saponin using the LC-ESI-MS methodology described above for the mesocarp saponins. The HPLC experiments were carried out by a reversed-phase C18 column and an isocratic 70:30 methanol and water used as a mobile phase. Nine peaks were separated and detected using both RI and MS detectors. Negative ion mode was operated in MS using ESI. The molecular ions in [M-H]⁻ of saponin peaks were observed and molecular weights were obtained. Fragmentations of each molecular ion were carried out by collision-induced dissociation (CID) experiments in order to identify the sugar chain and aglycone of the saponins.

From the nine peaks, five saponins with molecular mass of 1196, 1064, 1210, 1224 and 1078 were revealed. The mass spectrometric analysis of each nine peaks in negative ion mode gave deprotonated molecular mass from which molecular weights were calculated (Table 7).

The compounds of molecular mass 1196, 1064, 1046 and 1210 are the same saponins identified in the mesocarp extract. The saponin of MW 1224 (about 20% of the total saponins in the kernel cake) is composed of aglycone diosgenin, a Glue unit at position C26 and a second Rham unit bound to the second Glue unit in the C3 sugar chain, namely, a Gluc-Gluc-Rham; Rham chain instead of a Gluc-Gluc-Xyl; Rham chain in compound 1196. The saponin of MW 1078 (about 30% of the total saponins in the kernel cake) may be obtained by cleaving any one of the two Rham units of C3 sugar chain in compound 1224, thus, it contains either a Glue-Gluc-Rham or a Gluc-Gluc; Rham chain at position C3. Two isomers of the compounds of molecular mass 1046, 1078, and 1210 were identified based on their different retention times from the C18 column used for LC (Table 7).

TABLE 7
Major HPLC-RI peaks, retention time, amount and their corresponding
mass of methanol-extracted B. aegyptiaca seed kernel saponins

| | Retention | | |
|---|---|---|---|
| Peaks | time (min) | m/z [M − H]⁻ | amount (%)* |
| 1 | 5.4 | 1195 | 0.4 |
| 2 | 6.1 | 1063 | 0.6 |
| 3 | 7.5 | 1209 | 0.4 |
| 4 | 9.5 | 1223 | 6.7 |
| 5 | 10.9 | 1077 | 4.5 |
| 6 | 12.6 | 1209 | 11.2 |
| 7 | 14.6 | 1077 | 5.9 |
| 8 | 16.4 | 1045 | 2.0 |
| 9 | 23.3 | 1045 | 0.5 |

*Based on the injected sample

(iii) Chemical Characterization of Saponins Balanites Roots.

A methanolic extract of B. aegyptiaca roots of Example 2(ii) above was analyzed for saponin using the LC-ESI-MS methodology described above for the mesocarp and kernel cake saponins.

From the eleven peaks, nine saponins with molecular mass of 1196, 1064, 1210, 1224, 1340, 1516, 1530, 1572 and 1586 were revealed. Saponin with MW 1196 was the major saponin followed by saponin with MW 1224. Saponins having MW higher than 1500 Da were presented with minor peaks compared to the saponins with MW lower than 1500 Da. Saponin peaks with MW 1224 and 1530 were found twice.

The preliminary information about the major compounds with their mass in the methanol extract of B. aegyptiaca roots was obtained from the direct injection of the extract to the ESI-MS. However, for the comprehensive study, the mass spectrometric analysis of each of the eleven peaks obtained from the RI was carried out in negative ion mode and gave deprotonated molecular mass from which molecular weights were calculated (Table 8).

The compounds of MW 1196, 1210 and 1064 are the same saponins identified in the mesocarp extract and that of MW 1224 is the same saponin identified in the root extract.

The CID spectrum of the saponin of MW 1340 showed several fragments, including a fragment of MW 1196 representing a loss of 144 Da. The elimination of 144 Da, corresponding to cleavage of the E-ring, is observed generally in furostanol saponins. Such type of cleavage may occur only after a cleavage of the glucose residue at position C26 and has been reported by Liang et al. (2002) and Liu et al. (2004) in a furostanol saponin of Asparagus cochinchinensis. Thus, it is concluded that compound 1340 is built of the aglycone diosgenin and either a Gluc-Gluc-Xyl; Rham; Gluc-Gluc or a Gluc-Gluc-Xyl; Rham-Gluc; Gluc chain at position C3. Although it was not identified, a similar compound with a glucose unit at position C26 (MW 1502) is most probably found in the extract.

Two isomers of the saponin of MW 1530 were identified based on their different retention times from the C18 column used for LC. The CID spectrum of both of them showed several fragments, including a fragment of MW 1386 representing a loss of 144 Da and corresponding to the cleavage of the E-ring. Thus, it is concluded that compound 1530 is a methylated and hydrated form of the aglycone diosgenin and has a Gluc-Gluc-Rham; Rham-Gluc; Gluc-Gluc chain at position C3. Although it was not identified, a similar compound with a glucose unit at position C26 (MW 1692 for the methylated and hydrated form) is most probably found in the extract.

The saponin of MW 1516 has a similar structure as compound 1530 but with a loss of a covalent methyl group (CH₂). The additional saponins of MW 1572 and MW 1586 do not contain a glucose unit at position C26, as shown by the CID spectrum, and 1586 is a methylated form of 1572. However, the fragmentation process of both compounds includes an elimination of the E-ring and a further loss of additional 42 Da, which is yet not explained, and thus their structure is not clear.

TABLE 8
Major saponin peaks of the methanolic extract of B. aegyptiaca roots,
corresponding retention time, area and mass charge ratio in negative
ion mode (m/z)

| | Retention | | |
|---|---|---|---|
| Peaks | time (min) | m/z [M − H]^{-*} | amount (%) |
| 1 | 4.2 | 1339 | 0.8 |
| 2 | 5.4 | 1195 | 23.6 |
| 3 | 6.1 | 1063 | 1.7 |
| 4 | 7.4 | 1209 | 0.8 |
| 5 | 9.3 | 1223 | 7.4 |
| 6 | 9.9 | 1529 | 0.6 |
| 7 | 10.7 | 1585 | 0.7 |
| 8 | 11.6 | 1571 | 0.4 |
| 9 | 12.5 | 1223 | 5.1 |
| 10 | 13.6 | 1515 | 0.6 |
| 11 | 14.5 | 1529 | 0.4 |

*Integers obtained by truncating (not rounding off) of measured values

(ii) Chemical Characterization of Saponins from Balanites Oil.

LC-MS analysis of the saponin compounds extracted from the Balanites oil revealed saponin compounds, all based on the sapogenin aglycone diosgenin of MW 414 or 432 (414+18, for one molecule of H₂O), with a gradually growing sugar chain, including the saponins of MW 576 or 594 (414 or 432+162 for one Gluc unit); MW 738 or 756 (576 or 594+162 for another Gluc unit); MW 900 or 918 (738 or 756+162 for a further Gluc unit); MW 1046 or 1064 (900 or 918+146 for a Rham unit); MW 1178 or 1196 (1046 or 1064+132 for a Xyl unit). It seems that these compounds were obtained as a result of a cleavage process occurred during the oil extrusion.

(v) Biogenetic Saponin Pathway in B. aegyptiaca.

Based on the various LC-ESI-MS results obtained it can be concluded that all the saponin compounds extracted from Balanites aegyptiaca and identified herein consist of a sapogenin aglycone diosgenin of MW of 414, illustrated schematically in Scheme 1, with a glucose unit optionally linked at position C26 and a sugar chain optionally linked at position C3.

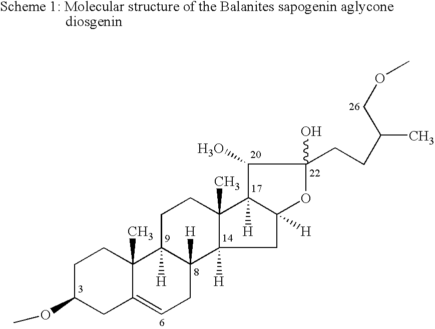

During the fragmentation process of all these saponin compounds, occurring either naturally in the Balanites tissues or during the extraction process, the C3 sugar chain and optionally also the C26 glucose unit are cleaved, generating various derivatives of the basic aglycone. Based on the CID spectrum of the LC-ESI-MS/MS of each one of the compounds, it can be concluded that all these compounds may be cleaved by loosing any terminal sugar moiety at a time. Namely, the C26 glucose unit may be cleaved while any sugar chain is still linked at position C3, and the cleaving order of the C3 sugar chain does not necessarily correlate the building process of this chain (biogenetic pathway A).

At any stage during the building process or the fragmentation process of the saponin compound, a molecule of H₂O may be attached to the aglycone diosgenin moiety or detached from it. Since this hydration occurs naturally in the Balanites tissues, a whole additional series of compounds may be identified, corresponding to the compounds obtained in the biogenetic pathway A with the addition of 18 for one H₂O molecule (biogenetic pathway B). In addition, due to the extraction with methanol, a well-known methanolysis reaction may occur at any stage, generating two additional series of compounds (biogenetic pathways C and D) corresponding to the molecules obtained in biogenetic pathways A and B with the addition of 14 for a covalent methyl group (CH₂) at position C22.

Furthermore, in case no glucose unit is bound to position C26, an elimination of 144 Da may occur at any stage during the fragmentation process, corresponding to the cleavage of the E-ring, thus, generating a further series of compounds in which the E-ring of the aglycone has been cleaved.

As mentioned above, several unidentified high-molecular saponins have been observed in the B. aegyptiaca root extract. Based on their CID spectrum, it is concluded that these compounds undergo a fragmentation process in which the E-ring is eliminated and a further unexplained loss of additional 42 Da occurs, thus their structure is not clear.

Table 9 presents the various configurations of the sugar chains at position C3 of the aglycone. Most of these sugar chains have been identified in the B. aegyptiaca saponins in accordance with the present invention. This table further presents the MW of each one of the compounds with or without the glucose unit at position C26 (biogenetic pathway A), as well as the MW of all the corresponding hydrates and methylated derivatives (biogenetic pathways B and C, respectively). As mentioned above, in case the C26 glucose unit has been cleaved, an elimination of 144 Da may occur corresponding to the cleavage of the E-ring.

TABLE 9
Balanites aegyptiaca saponins C3 sugar chain configurations and
molecular weights of corresponding compounds, hydrates and
methylated forms, with or without a C26 Glucose unit

| | | Molecular Weight | | |
|---|---|---|---|---|
| | | | | Methylated |
| | | | Hydrated form | form |
| | | With/without | with/without | with/without |
| No. | C3 Sugar Chain | C26 Glucose | C26 Glucose | C26 Glucose |
| 1 | | 576/414 | 594/432 | 590/428 |
| 2 | Gluc- | 738/576 | 756/594 | 752/590 |
| 3 | Gluc-Gluc- | 900/738 | 918/756 | 914/752 |
| 4 | 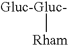 | 1046/884 | 1064/902 | 1060/898 |
| 5 | 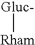 | 884/722 | 902/740 | 898/736 |
| 6 | 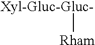 | 1178/1016 | 1196/1034 | 1192/1030 |
| 7 | 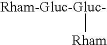 | 1192/1030 | 1210/1048 | 1206/1044 |
| 8 | Xyl-Gluc-Gluc- | 1032/870 | 1050/888 | 1046/884 |
| 9 | Rham-Gluc-Gluc- | 1046/884 | 1064/902 | 1060/898 |
| 10 | 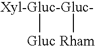 | 1340/1178 | 1348/1196 | 1354/1192 |
| 11 | 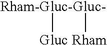 | 1354/1192 | 1372/1210 | 1368/1206 |
| 12 | 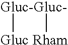 | 1208/1046 | 1226/1064 | 1222/1060 |
| 13 | 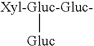 | 1194/1032 | 1212/1050 | 1208/1046 |
| 14 | 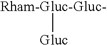 | 1208/1046 | 1226/1064 | 1222/1060 |
| 15 | 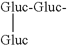 | 1062/900 | 1080/918 | 1076/914 |
| 16 | 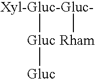 | 1502/1340 | 1520/1358 | 1516/1354 |
| 17 | 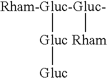 | 1516/1354 | 1534/1372 | 1530/1368 |
| 18 | 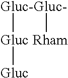 | 1370/1208 | 1388/1226 | 1384/1222 |
| 19 | 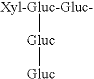 | 1356/1194 | 1374/1212 | 1370/1208 |
| 20 | 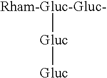 | 1370/1208 | 1388/1226 | 1384/1222 |
| 21 | 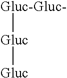 | 1224/1062 | 1242/1080 | 1220/1076 |
| 22 | 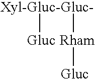 | 1502/1340 | 1520/1358 | 1516/1354 |
| 23 | 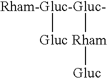 | 1516/1354 | 1534/1372 | 1530/1368 |
| 24 | 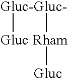 | 1370/1208 | 1388/1226 | 1384/1222 |
| 25 | 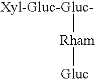 | 1340/1178 | 1358/1196 | 1354/1192 |
| 26 | 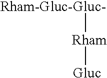 | 1354/1192 | 1372/1210 | 1368/1206 |
| 27 | 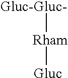 | 1208/1046 | 1226/1064 | 1222/1060 |
| 28 | 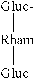 | 1046/884 | 1064/902 | 1060/898 |
| 29 | 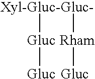 | 1664/1502 | 1682/1520 | 1678/1516 |
| 30 | 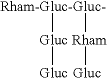 | 1678/1516 | 1696/1534 | 1692/1530 |

### Example 5

### Balanites aegyptiaca Saponin Nanovesicles

### (i) Balanites Saponins Nanovesicle Production.

Mixing plant saponins in aqueous solution, in a concentration that is above the critical micelle concentration (CMC), results in the formation of nanovesicles, which can encapsulate biological or chemical materials.

For the preparation of B. aegyptiaca saponin nanovesicles encapsulating biological or chemical materials, two main methods were used:

A) 100-200 μl saponin dissolved in methanol (approximately 0.008 gr/10 ml methanol) were injected into 2-4 ml of an aqueous solution which contained the compound/material to be encapsulated. The mixture was sonicated in a bath sonicator (Branson 2510) at 60° C. for short (1, 2, 5, 10 min) or long (60 min) periods, depending on the desired vesicle size and uniformity (Deamer and Bangham, 1976).

B) The saponin dissolved in ethanol or methanol (250-1000 μl) was added to a 50 ml round-bottom flask and the solvent was evaporated in vacuum until a thin oily film was formed on the flask bottom. An aqueous solution containing the compound/material intended to be encapsulated (1-4 ml) was added to the flask and the mixture was sonicated in a bath sonicator for 1-5 min at 60° C. (Szoka and Papahadjopoulos, 1978; Gregoriadis et al., 1998).

The size of the Balanites saponin nanovesicles varied between tens to hundreds of nm. Long sonication period combined with nanofiltration in a micro-extruder system enabled to control the vesicle size and to produce relatively uniform nanovesicles.

Nanovesicles from other saponins such as saponins extracted from Quillaja saponaria bark were prepared in the same way.

### (ii) Balanites Saponin Nanovesicle Microscopy Characterization.

The saponin nanovesicles preparations were characterized using light scattering measurements and various microscopic techniques including Transmission Electron Microscopy (TEM), Scanning Electron Microscopy (SEM), Light Scanning Electron Microscopy (WetSEM) and Atomic Force Microscopy (AFM).

The particle size of the saponin nanovesicles was measured using light scattering measurement techniques on ALV-NIBS High Performance Particle Sizer (ALV). Measurements were performed at an angle of 173°, λ=632 nm and 25° C. The light source was an argon ion laser and the photoelectron count-time autocorrelation function was calculated with a BI2030AT digital correlator (Brookhaven Instruments) and analyzed using the method of cumulants or the constrained regularization algorithm CONTIN applying the Stokes-Einstein relationship to the translational diffusion coefficients provides an intensity weighted distribution of hydrodynamic sizes (Finsy, 1994).

TEM experiments were carried out on either EM 201C (Phillips) or JEM-1230 (JEOL) using negative staining technique, employing saturated uranyl acetate solution (after centrifugation). The grid (300 mesh copper Formvar/carbon) was immersed in the vesicles solution for 1.5 minutes, stained in the uranyl acetate solution for 1.5 minutes, and then dried at room temperature on Whatmann filter paper (Johnsson and Edwards, 2000; Ottaviani et al., 2000).

SEM experiments were carried out in a Jeol-5600 Scanning Electron Microscope. Five μl of the vesicles solution were placed on a membrane and left at room temperature till full dryness. The membrane with the vesicles on its surface were glued on SEM stabs and coated with gold under vacuum.

In order to prepare the samples for Atomic Force Microscopy (AFM) experiments, 10 μl of the vesicles solution were placed on a freshly cleaved mika surface. After 2 min they were rinsed with 1-2 ml water, dried with a stream of nitrogen and further dried in a desiccator (Hansma et al., 1996; Thomson et al., 1996).

Spherical monolayer nanovesicles were clearly observed in the TEM, SEM and AFM images. The size of the nanovesicles varied between tens to hundreds of nm. Long sonication period combined with nanofiltration in a microextruder system enabled to control the vesicle size and to produce relatively uniform vesicles.

In order to evaluate the vesicle's stability, the saponin nanovesicle preparations were left at room temperature. The size, shape, membrane integrity, aggregation, and fusion between vesicles were examined by means of TEM as described above, once a week during 21 days. Only at the third week of the incubation period some fusion and aggregation of vesicles appeared, indicating a relatively high stability of the nanovesicles.

The Balanites saponin nanovesicles clearly and relatively efficiently encapsulated uranyl acetate as was demonstrated in the TEM images. In addition, as was shown in the TEM images, increasing the concentration of saponin in the preparation mixture caused a critical micelle concentration (CMC)-like effect (Demus et al, 1999). Initially, spherical vesicles organization was observed, but after a further increase of the saponin concentration, the structure of the vesicles was changed over rod-like structure and cubic-like structure. These assembly changes, related to the increase in the concentration of amphiphatic saponins in aqueous medium, suggest a lyotropic reaction that may be explained as a crystalline like phenomena (Demus et al, 1999). As far as we know, this phenomena was not yet reported for saponin compounds.

### Example 6

### B. aegyptiaca Nanovesicles with Encapsulated Bti Toxin

The Bti toxin was encapsulated in the Balanites saponin by both procedures A and B described in Example 5(i). The confocal experiments for visualization of the ability of Balanites saponin preparation to encapsulate Bti toxin were carried out on Zeiss LMS510. Five μl of the vesicles solution were placed on a microscope slide and left at room temperature till full dryness. The solution was prepared by adding 6.68×10⁻⁶ M rhodamine solution or 2×10⁻⁶ g/ml fluorescein solution to the oily film on the flask bottom and the mixture was sonicated in a bath sonicator for 1-5 min. Bti formulation (5 n\tilde{g}/ [symbol] stained with FITC (ULYSIS® Alexa Fluor® 488 Nucleic Acid Labeling Kit) (Molecular Probes, Leiden, The Netherlands) or rhodamine (tetramethylrhodamine-5-2′-deoxy-uridine-5′-triphosphate) were added to the solution. Twenty μl of vesicle's solution were spread on microscope slide and dried at room temperature (Schmidt et al., 1998). The concentrations of the solutions were determined experimentally.

Encapsulation of Bti toxin in Balanites saponin nanovesicles protected the toxin against inactivation and enabled to significantly extend its larvicidal activity for about 14 days. The Balanites saponin encapsulation afforded a clear LTV protection to the Bti toxin. In addition, the natural larvicidal activity of the Balanites saponins themselves provided a better larvae control results for a relatively long period of time. Moreover, the amphipatic nature of the saponin enabled to prevent the Bti from fast sinking to the lower water column and to maintain the product in the upper column at the larvae-developing zone. This long lasting anti-larvae activity reduces significantly the cost of the Bti treatment and provides better environment protection from the health risks caused by mosquitoes.

The activity of saponins from various sources, namely, saponins from Balanites aegyptiaca mesocarp extract (ME) or roots extract (RE) and from Quillaja saponaria commercial bark extract (QE, marketed by Sigma Co.), was tested on mosquito larvae. The effect of the saponins alone (550 mg/l) or formulated with Bti (0.02 mg/l) on two types of common mosquito larvae (Culex pipiens and Ades aegypti) at the same development stage (3^{rd}-4^{th} instars) was tested under control laboratory conditions and under sunlight exposure for various periods of time. The mortality of the larvae was recorded every day and new larvae in the same developmental stage (3^{rd}-4^{th} instars) were added to the treatment medium that was kept for the entire trials period. Tap water was used as control.

The results as summarized in Tables 10-11 clearly demonstrate the long lasting effect of Bti encapsulated in the saponin vesicle system, as well as the fast inactivation of Bti toxin under sunlight and the moderate larvicidal activity of the saponin itself.

TABLE 10
Effect of Bti encapsulated in B. aegyptiaca mesocarp (ME) or
in Q. saponaria (QE) saponin nanovesicles, with and without
sun exposure, on the mortality of Culex pipiens mosquito
larvae (3^{rd}-4^{th} instars)

| | % Mortality* | | | | | |
|---|---|---|---|---|---|---|
| Treatments | 1 day | 2 day | 3 day | 4 day | 5 day | 7 day |
| No sun exposure | | | | | | |
| Control (tap water) | 0 | 2 | 3 | 5 | 10 | 10 |
| Bti | 100 | 100 | 100 | 100 | 100 | 100 |
| QE | 0 | 3 | 13 | 13 | 28 | 48 |
| Bti + QE | 95 | 98 | 100 | 100 | 100 | 100 |
| ME | 2 | 20 | 38 | 53 | 70 | 80 |
| Bti + ME | 100 | 100 | 100 | 100 | 100 | 100 |
| During 7 days of | | | | | | |
| sun exposure of | | | | | | |
| the product | | | | | | |
| Control (tap water) | 0 | 0 | 5 | 10 | 10 | 10 |
| Bti | 0 | 0 | 0 | 0 | 5 | 10 |
| QE | 3 | 5 | 15 | 20 | 30 | 45 |
| Bti + QE | 38 | 58 | 63 | 73 | 78 | 85 |
| ME | 0 | 5 | 5 | 15 | 25 | 30 |
| Bti + ME | 80 | 95 | 97 | 98 | 100 | 100 |

*Values are the mean of 3 experiments with 20 larvae in each treatment each time.

TABLE 11
Effect of Bti encapsulated in B. aegyptiaca mesocarp or roots
(ME, RE) or in Q. saponaria (QE) saponin nanovesicles on the
mortality of Ades aegypti mosquito larvae (3^{rd}-4^{th} instars)

| | % Mortality* | | | | | |
|---|---|---|---|---|---|---|
| Treatments | 1 day | 2 day | 3 day | 4 day | 5 day | 7 day |
| No sun exposure | | | | | | |
| Control (tap water) | 7 | 8 | 12 | 20 | 22 | 22 |
| Bti | 100 | | | | | |
| QE | 12 | 13 | 13 | 16 | 28 | 48 |
| ME | 14 | 20 | 38 | 53 | 70 | 87 |
| RE | 17 | 41 | 69 | 100 | | |
| Bti + QE | 75 | 93 | 98 | 98 | 98 | 100 |
| Bti + ME | 100 | | | | | |
| Bti + RE | 100 | | | | | |
| After 4 days of | | | | | | |
| sun exposure of | | | | | | |
| the product | | | | | | |
| Control (tap water) | 2 | 12 | 12 | 12 | 12 | 12 |
| Bti | 0 | 0 | 0 | 2 | 5 | 10 |
| QE | 15 | 35 | 60 | 71 | 80 | 83 |
| ME | 19 | 26 | 42 | 65 | 75 | 79 |
| RE | 45 | 82 | 98 | 100 | | |
| Bti + QE | 28 | 38 | 43 | 43 | 43 | 43 |
| Bti + ME | 80 | 97 | 98 | 100 | | |
| Bti + RE | 95 | 100 | | | | |
| After 12 days of | | | | | | |
| sun exposure of | | | | | | |
| the product | | | | | | |
| Control (tap water) | 6 | 8 | 14 | | | |
| Bti | 0 | 0 | 0 | | | |
| QE | 24 | 41 | 57 | | | |
| ME | 39 | 46 | 59 | | | |
| RE | 52 | 71 | 83 | | | |
| Bti + QE | 51 | 68 | 74 | | | |
| Bti + ME | 73 | 84 | 89 | | | |
| Bti + RE | 84 | 96 | 100 | | | |

*Values are the mean of 3 experiments with 20 larvae in each treatment each time.

### Example 7

### Saponins as a Natural Bioadjuvant for Delivery of Agromaterials Through Plant Cuticular Membranes

The plant cuticle membrane is the primary barrier to plant uptake of pesticides and various agromaterials. To enhance the delivery and increase the penetration of pesticides, various synthetic surfactants and/or adjuvants are commonly used. However, due to phytotoxicity and potentially severe environmental side effects, many questions have been raised about these products due to their synthetic nature. Since saponins are fairly safe and easily biodegradable natural products, saponin-based delivery systems for agrochemicals provide an answer to the environmental issue raised by the use of synthetic adjuvants.

Although various saponin rich extracts have commonly been used in agriculture for their different activities but there has no report about the use of these extracts as an agricultural adjuvant. Considering the use of saponin in vaccine delivery and protective activities of saponins, we surmised that the amphiphilic saponin my also be used as a non-ionic, environmentally safe bio-adjuvant for foliar application of agrochemicals.

As a model test system we have chosen the most common saponin used in veterinary medicines, Quillaja saponaria extract with triterpenoid saponin as the main bioactive compound (Hostettmann and Marston, 1995) and the extracts of the Balanites aegyptiaca. with steroidal saponin as the main bioactive compound.

### Materials and Methods

(i) Preparation of the Saponin Rich Extracts

The Quillaja saponin preparation (QE) was made by dilution of commercial saponin extract of Q. saponaria bark (Sigma Aldrich, USA). Three Balanites saponin preparations were made by dilution of the methanol extracts of fruit mesocarp (ME), kernel extract (KE) and the root extract (RE) of B. aegyptiaca plant. The extracts were further defatted by petroleum ether (b.p. 60-80° C.). The defatted extracts were further eluted by methanol in solid phase extraction (SPE) cartridges (C 18, 35 ml, 10 g) after discarding first elution by water for removal of free sugars. The content of total sapogenins averaged 22-27% in all B. aegyptiaca extracts determined as described by Baccou et al. (1977). The saponin content of the QE was ≧25% (according to the manufacturer's specifications).

(ii) Leaf Cuticle Isolation

Full-grown matured leaves of the Citrus grandis L. having astomatous cuticles were collected from the C. grandis plant grown in the commercial orchard of the Kibutz Yad-Mordechai (Israel) and washed in de-ionized distilled water (DDW). After punching discs of 20 mm diameter using cork-borer out of the leaves, the cuticles were isolated enzymatically by incubating the leaf discs in a mixture (1:1) of cellulose 203-13 L (Biocatalysts, UK) and Pectinase 62 L (Biocatalysts, UK) as described by Schonherr and Riedererm (1986) at concentration of 1% (w/w) of citric acid buffer (0.1M) at 40° C. and pH 4. After a few days, astomatous cuticles from the upper leaf epidermis were collected, rinsed extensively and desorbed in DDW, air-dried on Teflon discs and stored in refrigerator until used. These isolated astomatous adaxial cuticles are referred hereafter as cuticular membrane (CMs).

(iii) Delivery Experiments

In the first phase of the study, four donor solutions (1% of each QE, ME, KE and RE) were prepared by adding ¹⁴C-labeled 2,4-D (specific activity 19.2 mCi/mmol, Sigma) as a tracer (30 000-40 000 cpm/μl) and compared with both negative (DDW) and positive (1% Triton X-100, Sigma) control. Rates of the cuticular penetration were measured at 30% RH and 30° C. using SOFU procedure as described by Schonherr (2000) and modified by Wiesman et al. (2002). A special delivery system with a thermostat desorption chamber and a controlled environment was designed for the experiment. CMs were mounted between the lid and bottom of desorption chambers using silicon grease (Bayer, Germany). Each CM was tested for leaks. DDW was added to desorption chamber for 24 h. After 24 h the DDW was withdrawn and 10 μl droplet of donor solutions (1% solution of QE, ME, KE, RE and DDW with 2,4-D [¹⁴C] was placed on the center of the CM. After water was evaporated from the donor solution, the chambers were filled again with DDW, which serves as receiving solution.

Receiver solution (DDW) was quantitatively withdrawn after 1, 4, 24, 48, 72, 96, 120, 144 and 168 h of the experiment for scintillation counting and was replaced by fresh ones. At the end of the experiment, the CM was cut out and, after adding scintillation cocktail, counted to determine the amount of radioactive material left of the surface of the CM. A Beckman LS 1701 scintillation counter (Beckman Coulter, USA) was used to determine radioactivity of samples. The amount applied (M₀) was calculated by summing amounts penetrated (Mₜ) plus the amount left on the CM at the end of the experiment in individual CM. Thus Mₜ/M₀ is the fraction that penetrated and 1−(Mₜ/M₀) is the fraction still left on the surface of the CM. Data were plotted as −ln (1−Mₜ/M₀) vs. time as described by Schonherr (2000). The experiment was repeated three times.

In the second phase of the study, six concentrations of the ME (0.1, 0.25, 0.5, 1.0, 2.0 and 5% w/v) was tested as donor solutions by adding ¹⁴C labeled 2,4-D and compared with the negative control (DDW). The preparation of both donor and receiver solution and the methods of experiment were same as first phase of the study. In this phase of study the experiment was also repeated three times.

In the third phase of the study, the effect of the penetration of 1% ME was compared in different humidity (30, 60 and 90% RH) and temperatures (30, 45 and 60° C.). While comparing humidity, the temperature was set at 30° C. and during the temperature study humidity was set for 30% RH. In each experiment at least 20 CMs were used in each treatment.

(iv) Microscopic Study

TEM characterization of the different saponin rich solutions were carried out on JEOL-JEM-1230 Electron microscope (Japan) using negative staining technique, employing saturated uranyl acetate solution (after centrifuge) using Ultra-pure water (Biological Industries, Israel). The grid (300 mesh copper Formvar/carbon) was immersed in the 1% solution of each ME, RE, KE and QE for 1.5 minutes and then stained in the uranyl acetate solution for 1.5 min. The grid then dried in room temperature on Whatmann filter paper (Ottaviani et al., 2000). The dried grids were examined at 8000 KV accelerating voltage and 25 K magnification.

Particle sizes of the extracts were measured using light scattering measurements techniques on ALV-NIBS High Performance Particle Sizer (Germany). Each saponin extracts (ME, KE, RE and QE) were prepared in 0.5% by using Ultra-pure water (Biological Industries, Israel) in a dust free condition. Measurements were performed at an angle in 173° at 632 nm λ in 25° C. The light source was an argon ion laser and the photoelectron count-time autocorrelation function was calculated with a BI2030AT (Brookhaven Instruments) digital correlator and analyzer using the method of cumulants or the constrained regularization algorithm CONTIN applying the Stokes-Einstein relationship to the translational diffusion coefficients provides an intensity weighted distribution of hydrodynamic sizes (Finsy, 1994)

(v) Statistical Analysis

Statistical analysis for the data was performed with JMP software (SAS, 2000) using the Tukey-Kramer HSD test for determining significant difference among treatments at P=0.05 level of significance.

### Results

### 7(i) Effect of the Saponin Source on Penetration of 2,4-D Across the CMs

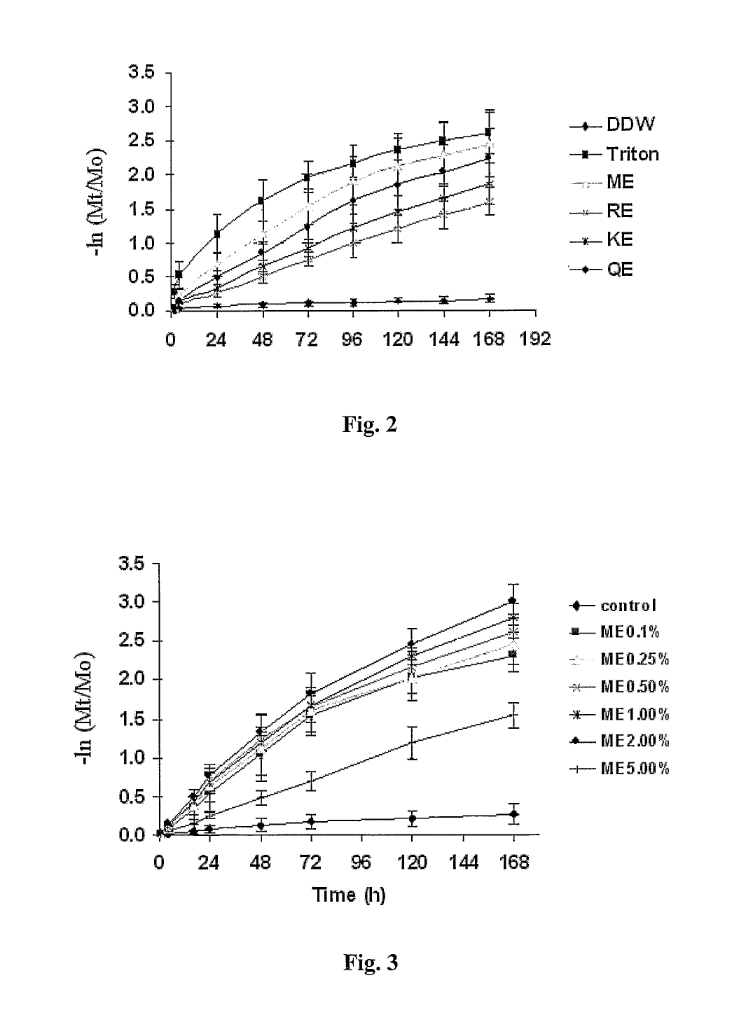

The effect of the different saponin sources on the penetration of the 2,4-D (¹⁴C) is presented in FIG. 2. The rate penetration of 2,4-D was highest initially, but tended to level off with time. Hence, the penetration of the 2,4-D can be completely described by a single constant, the rate constant (k) of penetration, which is equivalent to the slope of the straight line (Schonherr, 2000). When no adjuvant was added in 2,4-D (control-DDW), the rate constant was 0.595×10⁻⁵ h⁻¹. The penetration rate was 9.8 times higher (5.833×10⁻⁵ h⁻¹) when RE was used. Similarly, a 10.5 times higher (6.18×10⁻⁵ h⁻¹) and 14.7 times higher (8.750×10⁻⁵ h⁻¹) penetration rates were achieved in KE and QE treatment whereas a 17.2 times higher (10.20×10⁻⁵ h⁻¹) penetration rate was achieved in ME treatment, and 19.1 times higher (11.000×10⁻⁵ h⁻¹) in Triton treatment. Among all the saponin extracts tested, highest penetration rate of 3,4-D was achieved in ME. This rate of penetration was not significantly different when compared with the commercial Triton X-100 surfactant (a positive control) (Table 12).

TABLE 12
The effect of the saponin as an adjuvant on the penetration rate of
2,4-D (¹⁴C) across the Citrus grandis leaf CMs at 30° C. and 30% RH

| | Penetration | Factor increase |
|---|---|---|
| Treatment | rate (h⁻¹) | (times) |
| DDW | 0.59 × 10⁻⁵ d | 0.0 |
| RE | 5.83 × 10⁻⁵ c | 9.8 |
| KE | 6.18 × 10⁻⁵ d | 10.5 |
| QE | 8.75 × 10⁻⁵ b | 14.7 |
| ME | 10.20 × 10⁻⁵ a | 17.2 |
| Triton X-100 | 11.00 × 10⁻⁵ a | 18.6 |

Triton, QE, ME, KE, RE refer to 1% (w/v) solution of Triton X-100, Q. saponaria extract saponin, B. aegyptiaca fruit mesocarp extract saponin, B. aegyptiaca kernel extract saponin and B. aegyptiaca root saponin extract. DDW refers to deionized distilled water and contained only 2,4-D solution as a control. Each value is the mean of the pool data of 60 CMs. Means sharing common postscripts are not significantly different (P < 0.05).

### 7(ii) Effect of the Concentration of the ME on Penetration of 2,4-D Across CMs

The first phase of study showed that all saponin rich extracts enhanced the penetration of 2,4-D across the CMs. Since there was no significant difference in rate penetration between ME and the commercial foliar adjuvant Triton, in the second phase of the study different concentrations of ME were tested. Thus, six ME concentrations (0.1, 0.25, 0.5, 1.0, 2.0 and 5.0%) were tested as donor solutions by adding to ¹⁴C-labeled 2.4-D and compared with the negative control (DDW).

As shown in FIG. 3, the penetration rate through the CMs increased with the ME concentration from 0.1% to 2.0% and was the highest with 2% Me (11.9×10⁻⁵ h⁻¹), which was almost 20 times higher than the penetration rate of the control treatment (DDW), which was just 0.59×10⁻⁵ h⁻¹. However, the penetration rate of 1% ME was not significantly different from 2% ME and drastically decreased when 5.0% ME was used (5.65×10⁻⁵ h⁻¹) (Table 13).

TABLE 13
The effect of various concentrations of B. aegyptiaca fruit mesocarp
saponin extract (ME) as an adjuvant on the penetration rate of
2,4-D (¹⁴C) across the Citrus grandis leaf CMs at 30° C. and 30% RH

| | Penetration | Factor increase |
|---|---|---|
| Treatment | rate (h⁻¹) | (times) |
| DDW | 0.59 × 10⁻⁵ f | 0.0 |
| ME 0.10% | 9.46 × 10⁻⁵ c | 15.8 |
| ME 0.25% | 9.88 × 10⁻⁵ bc | 16.7 |
| ME 0.5% | 10.53 × 10⁻⁵ b | 17.8 |
| ME 1.0% | 11.07 × 10⁻⁵ a | 18.7 |
| ME 2.0% | 11.90 × 10⁻⁵ a | 20.0 |
| ME 5.0% | 5.65 × 10⁻⁵ e | 9.5 |

Each treatment contained the same concentration of 2,4-D together with the reported concentration of ME as listed. Each value is the mean of the pool data of 60 CMs. Means sharing common postscripts are not significantly different (P < 0.05). DDW refers to deionized distilled water as a control.

7(iii) Effect of the Humidity and Temperature on Penetration of 2,4-D Across CMs

In the third phase of the study, the effect of humidity (30, 60 and 90%) and temperature (30, 45 and 60° C.) over the penetration rate of 1% ME across the CMs were tested. When comparing relative humidity, the temperature was set at 30° C. and in the temperature study, humidity was set at 30%. Increase of the humidity or of temperature resulted in increased penetration rate of 2,4-D (Table 14).

TABLE 14
The effect of humidity and temperature on the penetration rate of
2,4-D (¹⁴C) across the Citrus grandis leaf CMs, with 1% B. aegyptiaca
fruit mesocarp extract saponin (ME) as an adjuvant

| | |
|---|---|
| Treatments | Penetration rate (h⁻¹) |

Humidity (%)

| | |
|---|---|
| 30 | 10.46 × 10⁻⁵ (0.71 × 10⁻⁵) |
| 60 | 13.32 × 10⁻⁵ (2.45 × 10⁻⁵) |
| 90 | 16.35 × 10⁻⁵ (4.36 × 10⁻⁵) |

Temperature (° C.)

| | |
|---|---|
| 30 | 10.35 × 10⁻⁵ (0.59 × 10⁻⁵) |
| 60 | 12.32 × 10⁻⁵ (2.44 × 10⁻⁵) |
| 90 | 16.15 × 10⁻⁵ (4.76 × 10⁻⁵) |

The values in parenthesis are the penetration rates of the control in respective treatment in respective conditions. Humidity experiment was conducted in 30° C. whereas temperature experiment was conducted in 30% humidity

### 7(iv) Light Scattering Characterization of the Particle Size Distribution and TEM Characterization of the Saponin Solution

The average diameter of the mean mass particle population of the ME, KE, RE and QE solution (1%) was characterized. The mean diameter of the ME and QE solution were 167 nm and 177 nm whereas average diameter of the particle of the mass population of KE and RE were almost three times higher, i.e., 502 nm and 587 nm, respectively.

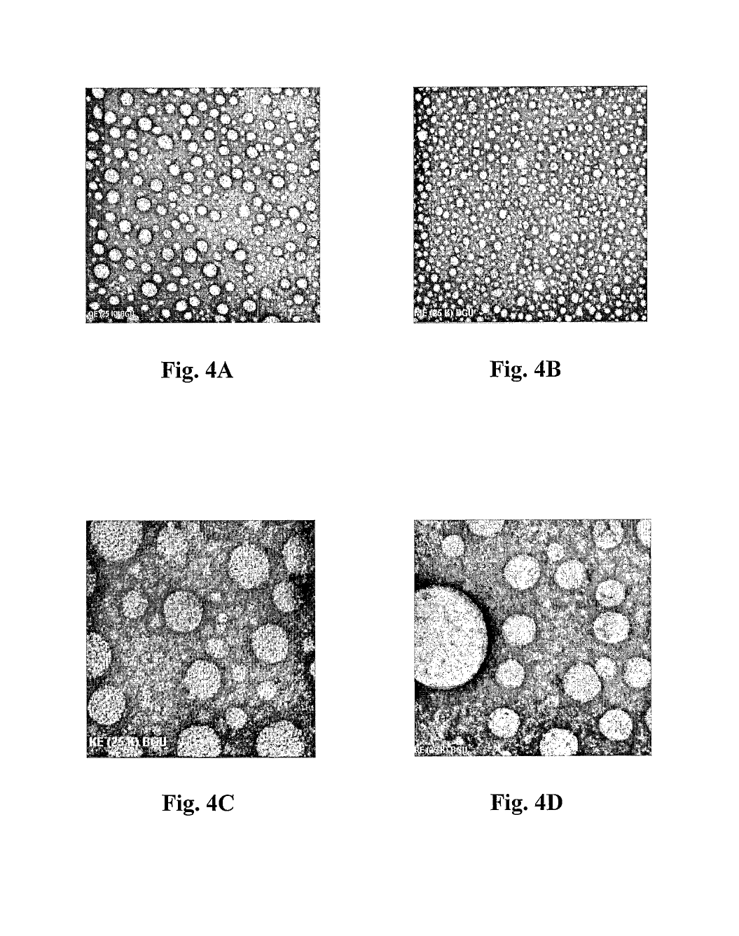

When all four saponin rich solutions (QE, ME, KE and RE) that were used earlier in different penetration experiments were characterized in TEM, small nanosized vesicles were observed in all four solutions (FIGS. 4A-D). The size and structure of the vesicles in both QE and ME had similarity whereas both the size and structure of the vesicles in RE and KE were slightly different than the former ones. In QE and ME the shape of the nanosized vesicles was spherical whereas some rod shaped structures were also observed in RE (FIG. 4D).

One possible reason for the increase in the penetration rate of the agrochemical using the saponin as adjuvant is the formation of the nanovesicles or micelles that help the penetration. The microscopic study of the saponin solutions of the invention showed the formation of natural nanosized vesicles or micelles (FIG. 4). The saponin nanovesicles were collected after their delivery through the cuticle membrane and observed using TEM. The TEM image shows that the nanovesicles remained in their original nanovesicle shape, which indicates their stability and capacity to penetrate through biological membranes in an intact form.

### Example 8

### Balanites Saponin Nanovesicles Encapsulating Vitamin C

The effect of Balanites mesocarp saponin preparation on delivery of vitamin C through the skin was tested using isolated rat skin system. 100 μl of saponin nanovesicles containing encapsulated vitamin C (2 mg) were prepared from saponin extracted from Balanites mesocarp by the procedure A described in Example 5(i) above. 15 μl of the preparation were loaded on an isolated rat skin in three replicates. The treatments were applied in a controlled environment (25° C. and 60% relative humidity) and incubated for 30 or 60 min. The solution delivered through the isolated skin was tested using an HPLC system for detecting the level of vitamin C corresponding to a standard peak of vitamin C. The results, shown in Table 15, clearly demonstrate a significant increase in the penetration rate of non-oxidized vitamin C, compared to the control, in both tested incubation periods. This finding demonstrates again the protective effect provided by the Balanites saponin nanovesicle system to sensitive biomaterials.

TABLE 15
Effect of Balanites mesocarp saponin nanovesicles preparation on
vitamin C absorption through rat skin

Vitamin C level in skin
(ng/cm² skin surface area)

| | 30 minutes | 60 minutes |
|---|---|---|
| Treatment | incubation | incubation |
| Control (Vitamin C) | 259 ± 109 | 326 ± 131 |
| Vitamin C + ME | 595 ± 279 | 909 ± 166 |

### Example 9

### Saponins as Irrigation Water Adjuvants

Adding plant extract rich in saponins to water applied to the bases of mung bean cuttings significantly increased the number of roots formed and their length (Table 16). This effect was increased as saponin concentration increased up to 500 ppm for Balanites mesocarp extract saponins and 50 ppm for Balanites root extract saponins, and up to 100 ppm for Quillaja saponaria bark extract saponins. The treatment solution was replaced by fresh water after 24 h and the rooting was assessed 10 days after the initiation of the experiment. The stimulating effect of saponins on root formation may be related to their hormone-like effect, but their effect on root elongation seems to be mainly due to their influence on increase of water uptake.

The enhancing effect of saponins on rooting of mung bean cuttings when applied together with the synthetic plant hormone indole-3-butyric acid (IBA) is shown in Table 17. Adding 100 ppm of Balanites mesocarp and root extract saponins significantly increased the number of formed root and their development in comparison to untreated control and to the common hormonal treatment with IBA. Similar, but weaker, enhancing effect was found with Quillaja bark extract saponins (the treatment solution was replaced by fresh water after 24 h and the rooting was assessed seven days after the initiation of the experiment).

TABLE 16
The effect of saponin concentrations on the rooting of mung bean
cuttings

| | Number of | |
|---|---|---|
| | roots per | Average root |
| Treatment | cutting* | length* (mm) |
| Control (H₂0) | 7.6 ± 0.4^{d} | 22 ± 3.5^{d} |
| ME (10 ppm) | 7.5 ± 0.4^{d} | 23.7 ± 2.5^{cd} |
| ME (50 ppm) | 7.8 ± 0.6^{d} | 24.2 ± 1.7^{cd} |
| ME (100 ppm) | 8.9 ± 0.4^{cd} | 30.3 ± 1.4^{ab} |
| ME (500 ppm) | 14.2 ± 0.8^{a} | 32.7 ± 1.5^{a} |
| RE (10 ppm) | 8.6 ± 0.4^{cd} | 25.3 ± 3.2^{cd} |
| RE (50 ppm) | 13.5 ± 0.6^{ab} | 33. ± 2.9^{a} |
| RE (100 ppm) | 12.5 ± 0.5^{b} | 32.4 ± 2.3^{a} |
| RE (500 ppm) | 4.8 ± 0.5^{e} | 12.8 ± 1.9^{e} |
| SS (10 ppm) | 7.8 ± 0.5^{d} | 23 ± 1.2^{cd} |
| SS (50 ppm) | 8.5 ± 0.6^{cd} | 23.4 ± 2.3^{cd} |
| SS (100 ppm) | 12.8 ± 0.3^{b} | 28.3 ± 1.9^{b} |
| SS (500 ppm) | 8.2 ± 0.9^{cd} | 12.3 ± 2.7^{e} |

TABLE 17
Additive effects of saponins plus IBA on the rooting of the mung
bean cuttings

| | Number of | |
|---|---|---|
| | roots per | Average root |
| Treatment | cutting* | length* (mm) |
| Control (H₂0) | 7.1 ± 0.1^{d} | 12.0 ± 1.4^{bc} |
| IBA 100 μM | 28.3 ± 1.3^{c} | 10.1 ± 0.4^{cd} |
| ME 100 ppm + IBA 100 μM | 40.8 ± 1.2^{a} | 16.2 ± 1.5^{a} |
| RE 100 ppm + IBA 100 μM | 43.9 ± 0.7^{a} | 18.9 ± 0.7^{a} |
| SE 100 ppm + IBA 100 μM | 34.6 ± 0.8^{b} | 13.6 ± 0.3^{b} |

*Each value is the mean of 24 observations from the combined data of 12 replicates of two experiments ± SE. Means in each column followed by different letters are significantly different at P = 0.05. ME = B. aegyptiaca mesocarp extract saponins; RE = B. aegyptiaca root extract saponins; SS = Q. saponaria bark extract saponins.

In another experiment, the saponins were added to saline irrigation water. Low quality water, mainly containing high saline level, is used today for irrigation of a wide range of agriculture crops all over the world. Saline water is well known to inhibit the growth and development of the majority of crops. This inhibition effect is mainly due to the influence on osmotic potential and in some cases also due to toxicity of chloride ions. Irrigation of plants with increased saline level significantly reduces the water uptake by the root system, and therefore causes growth inhibition that leads in many cases to plant drying, depending on the plant species.

As shown in Table 18, mung bean seeds germination was retarded by increased level of NaCl in comparison to control of tap water 4 days after the beginning of germination. Addition of Balanites mesocarp extract (ME) saponins in moderate concentration of 75 mM to the irrigation saline water stimulated the germination and development of mung bean seeds in comparison to control. It also stimulated the germination of the seeds in comparison to control and to saline treatments. The effect on the foliage and the roots of the treated mung bean seedlings corresponded to the effect on the germination. These effects are directly related to the effect of Balanites mesocarp saponin treatment on water uptake by the mung bean seeds and germinated seedlings.

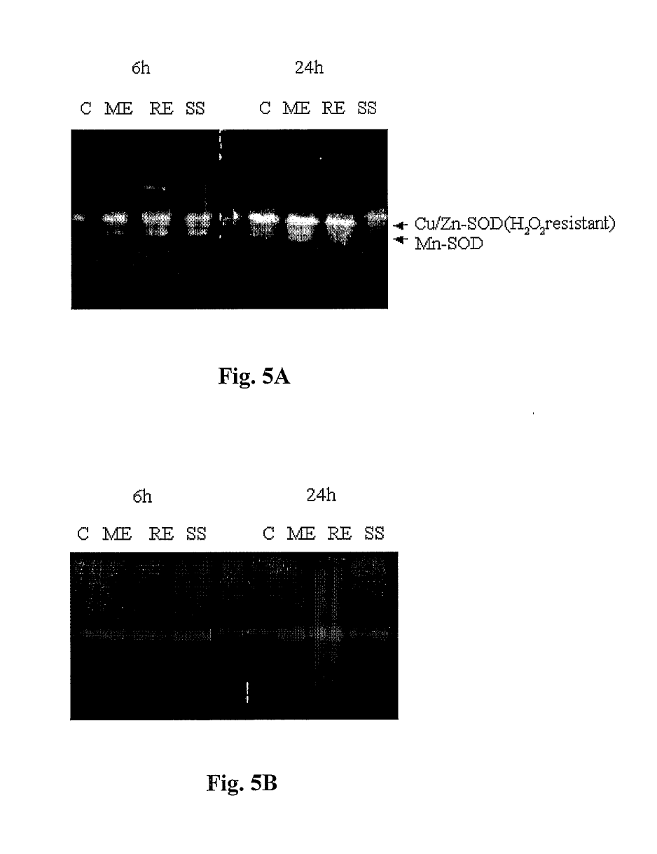

Due to the effect of saline water on osmotic pressure leading to reduction of water uptake by the plant roots and due to additional direct effects, the oxidative enzymatic system is activated. As shown in FIGS. 5A-5B, Balanites extract saponins were found to induce the enzyme superoxide dismutase (SOD) (E.C. 1.15.1.1), which is well known to be involved in the oxidative response to saline irrigation stress, but not the enzyme glutathione peroxidase (GSH-Px) (E.C. 1.11.1.9).

Total proteins were subjected to electrophoresis on non-denaturing polyacrylamide gels and stained directly for GSH-Px activity according to Lin et al (2002). In brief, the gel was submerged in 50 in NI Tris-HCl buffer (pH 7.9) containing 13 mM glutathione and 0.004% hydrogen peroxide, and gently shaken for 10-20 min. The GSH-Px activity was stained with 1.2 mM 3-(4,5-dimethylthiazol-2-yl)-2,5-diphenyltetrazolium bromide (MTT) and 1.6 mM phenazine methosulfate (PMS).

For SOD activity, non-denaturing polyacrylamide gels were stained with a mixture of 501-mM Tris-HCl buffer (pH 8.5), 1.2 mM MTT, 1.6 mM PMS and MgCl₂.6H₂O according to Brewer et al (1967) and visualized in daylight.

The SOD isoforms were identified by their differential sensitivities to KCN and H₂O₂: Cu/Zn-SOD is sensitive to both KCN and H₂O₂, Fe-SOD is sensitive only to H₂O₂, and Mn-SOD is resistant to both inhibitors (Okamoto and Colepicolo, 1998). Thus, during visualization, gels were incubated separately for 60 min in either 5 mM KCN or in 0.5 mM H₂O₂.

TABLE 18
Effect of Balanites mesocarp saponin preparation on mung bean
seedlings germination, growth and development under saline
water irrigation

| | Germination | Foliage | |
|---|---|---|---|
| Treatment | (4 days) | (14 days) | Root |
| Control | +++ | aaa | bbb |
| ME 75 mM | ++++ | aaaa | bbbb |
| ME 150 mM | +++ | aaa | bbb |
| NaCl 75 mM | ++ | aa | bbb |
| NaCl 150 mM | + | a | bb |
| NaCl 75 mM + ME 100 mM | +++ | aaaa | bbbb |
| NaCl 150 mM + ME 100 mM | ++ | aaa | bbb |

* The observations were based on development ladders as follow: typical germination using tap water +++ (1 cm height with two leave starting to be expended); ++++ more advanced seedling; ++ less advanced seedling; + not germinated seedling; Typical mung bean foliage development 14 days after germination using tap water aaa (~10 cm seedling with fully expanded two leave and abscised cotyledons); aaaa more elongated seedlings; aa less elongated seedlings; a dwarf seedling; Typical mung bean root system 14 days after germination using tap water bbb (~6 cm well branched roots); bbbb more elongated and branched roots; bb less elongated roots; b short and necrotic root system.

### Example 10

### Biocidal Activity of B. aegyptiaca Saponins in Aqueous Media

This Example presents the natural biocidal activity of Balanites saponins in three major biotic fields closely related to aqueous media: larvae control, fungi control and bacteria control.

### (i) Balanites Saponin Effect on Larvae Control.

During the last decade, the public awareness in Europe to problems caused by mosquitoes and mosquito-borne infectious viral diseases is increasing. Mosquitoes strongly affect human quality of life while interfering with human activities and the economy, particularly the tourist industry. Major tourist resorts in many European countries are plagued with both day-biting Aedes and night-biting Culex and Anopheles nuisance mosquitoes, whose populations peak during the tourist season (June-August).

Suppression of nuisance mosquitoes by means of traditional chemical insecticides is becoming more and more difficult due to the resistance mosquitoes have developed to toxic substances and the detrimental effect of chemical pesticides on the environment. Reduction or replacement of conventional chemical pesticide usage has come under increasing public pressure over the past several years, due to their environmental impact (large spectrum of toxicity) and sanitary risks (direct exposure of operators and public, contamination of water, ground and air).

Development and implementation of integrated biological control (IBC) in the EU countries is already in progress by adopting environmentally friendly approaches to reduce nuisance and vector mosquito population, by directing control efforts at the breeding site and reducing the need for ineffective and environmentally adverse mosquito adulticide applications.

The environmentally friendly biological control agent, which is playing a leading role in the field of mosquito control in Europe, is Bti. Bti is easy to produce in large scale and is widely considered the most selective available mosquito control agent. As described above, contrary to chemical pesticides with their wide range of toxic effects on the environment, Bti is selective in its larvicidal activity and highly specific to mosquito and blackfly larval populations only, with negligible effects on non-target invertebrate or vertebrate organisms.

As shown in Example 6, Bti toxin can be encapsulated and protected from UV irradiation in saponin nanovesicles and the larvicidal activity of Balanites saponin-Bti was maintained for about two weeks. However, several previous reports based on crude saponin mixtures suggested that saponins, by themselves, are active against larvae. We have, then, tested the effect of non-formulated purified Balanites mesocarp extract main saponin (MW 1064) on larvae control.

Eggs of laboratory-grown Aedes aegypti were hatched in tap water in a growth chamber with a 16-hour photoperiod, at a temperature of 25±3° C. Twenty to twenty five larvae at the late 3^{rd} and early 4^{th} instars were placed into 150-ml disposable plastic cups containing 100 ml of tap water together with water-soluble saponin solutions of various concentrations. During the experiments, treated and control (0 mg/L saponin) cups were examined after 2, 4, 9 and 11 days and number of alive larvae or adults were counted. The data are presented in percentage together with an analysis of variance using the Tukey-Kramer HSD test (SAS's JMP software) at the 0.01 level of significance.

As shown in Table 19, application of Balanites mesocarp main saponin to the larvae growing water in concentration of up to 100 ppm had very little control effect on larvae mortality and indeed relatively high percent of pupa and later adult mosquito were developed. However, in concentrations of 300 up to 800 ppm, the above-mentioned saponin showed increased larvae control already two days after application and provided almost full control of Aedes aegypti adult mosquitoes.

TABLE 19
Effect of Balanites main mesocarp saponin (MW 1064)
concentrations on Aedes aegypti mosquito larvae, pupa and adults

| | Days | | | | |
|---|---|---|---|---|---|
| Concentration (ppm) | 0 | 2 | 4 | 9 | 11 |
| % dead larvae | | | | | |
| 0 (control) | 0 | 0 d | 1.25 d | 2.5 d | 5 cd |
| 10 | 0 | 0 d | 0 d | 0 d | 1.25 d |
| 100 | 0 | 0 d | 0 d | 6.25 d | 6.25 cd |
| 300 | 0 | 50 c | 50 c | 50 c | 48.75 bc |
| 500 | 0 | 77.5 b | 78.75 b | 78.25 b | 78.25 ab |
| 800 | 0 | 95 a | 95 a | 93.75 a | 94.25 a |
| 1000 | 0 | 90 a | 94 a | 93.75 a | 96.25 a |
| % pupa | | | | | |
| 0 (control) | 0 | 0 | 25 abc | 0 b | 0 |
| 10 | 0 | 0 | 41 ab | 0 b | 0 |
| 100 | 0 | 0 | 45 a | 0 b | 0 |
| 300 | 0 | 0 | 15 bc | 33.25 a | 0 |
| 500 | 0 | 0 | 9.5 c | 1.75 b | 0 |
| 800 | 0 | 0 | 1.75 c | 0 b | 0 |
| 1000 | 0 | 0 | 3.75 c | 0 b | 0 |
| % adults | | | | | |
| 0 (control) | 0 | 0 | 0 | 62 ab | 84 a |
| 10 | 0 | 0 | 0 | 41 bc | 87 a |
| 100 | 0 | 0 | 0 | 70 a | 85 a |
| 300 | 0 | 0 | 0 | 15 d | 50 b |
| 500 | 0 | 0 | 0 | 20 cd | 22.5 c |
| 800 | 0 | 0 | 0 | 0 d | 5 c |
| 1000 | 0 | 0 | 0 | 3.75 d | 3.75 c |

*Means in each column followed by different letters are significantly different at P ≦ 0.01

### (ii) Balanites Saponin Effect on Fungi Control.

The inhibitory effect of Balanites purified main mesocarp saponin (MW 1064) on mycelial colony growth of Pythium aphanidermatum was tested in vitro.

Cultures of Pythium aphanindermatum were isolated from plant species showing Pythium symptoms. The isolate was placed in potato dextrose agar (PDA, Difco, Detroit, Mich., USA) supplemented with 100 ppm of streptomycin and incubated in the dark at 27° C. for 2 days. The colony of Pythium was identified visually and microscopically and sub-cultured on PDA without antibiotics as described by Tsror et al (2001).

For fungal growth inhibition measurements five concentrations of Balanites mesocarp saponin (ME) preparation (0.1, 0.5, 1.0, 2.0 and 4.0% w/v) and two concentrations of Metalaxyl-10 (a systemic benzenoid fungicide, 1 and 5 ppm as positive control) were added in separate Erlenmeyer flasks containing sterilized (121° C., 2 atm, 20 min) PDA, and mixed properly. Samples of PDA containing Balanites mesocarp saponin preparation and Metalaxyl-10 (15 ml) were poured into sterilized petri dishes separately and allowed to solidify. 5 mm in diameter agar plug of the fungal isolate was placed in the center of each plate. The plates were incubated at 27° C. (10 plates per treatment). The colony diameter of the mycelial growth was measured after 24 and 48 h and the inhibitory activity of each treatment was expressed as the growth inhibition percentage as compared to the negative control (0% ME preparation) using the formula: Growth inhibition percentage=[100-(Treatment growth/Control growth*100)].

The results, shown in Table 20, clearly demonstrate that Balanites purified main mesocarp saponin strong inhibition of Pythium aphanindermatum growth is similar to the effect of the conventional synthetic fungicide used as a positive control. Significant growth inhibition of the fungus was observed already in concentration of 1% Balanites mesocarp main saponin and the inhibition rate was concentration dependent.

TABLE 20
Growth inhibition of B. aegyptiaca purified main mesocarp saponin
(ME) on mycelial colony growth of Pythium aphanidermatum in vitro

| | % Growth inhibition* | |
|---|---|---|
| Type of Treatment | 24 h | 48 h |
| ME 0.1% | 4.29 ± 1.50 d | 1.00 ± 0.01 e |
| ME 0.5% | 13.96 ± 3.21 d | 5.35 ± 1.32 e |
| ME 1.0% | 66.39 ± 3.52 c | 26.01 ± 2.95 c |
| ME 2.0% | 81.96 ± 1.30 b | 70.87 ± 2.57 b |
| ME 4.0% | 82.47 ± 1.30 b | 80.01 ± 0.42 b |
| Metalaxyl (10%) 1 ppm | 83.53 ± 2.45 b | 61.12 ± 2.98 c |
| Metalaxyl (10%) 5 ppm | 100 ± 0.00 a | 100 ± 0.00 a |

*Means in each column followed by different letters are significantly different at P ≦ 0.01

(iii) Balanites Saponin Effect on Bacteria Control.

The effect of Balanites purified main mesocarp, kernel and roots extract saponins was tested on growth of Escherichia coli.

Standard Luria Broth solution (LB) was prepared using 5 g yeast extract, 10 mg NaCl and 10 mg Typtone per ml of DDW. A small portion of the Escherichia coil inoculum (LMC1492 strain, obtained from the Life Sciences Department of the Ben-Gurion University) was added to the LB solution in a sterilized Erlenmeyer and inoculated in a rotary water bath incubator at 250 rpm and 37° C. The tested extracts were first prepared in 5% in LB solution and used as stock solution for further dilution. Lower concentrations were obtained by mixing this stock solution in the required ratios with plain LB solution. The extract containing solution of the required concentrations were prepared in 10/140 mm glass test tubes in volumes of 1.8 ml each and incubated in a rotary water bath incubator (Gyvotory-676, New Brunswick Scientific Co. Inc, NY) at 250 rpm and 37° C. Bacterial cultures were added at 1:40 dilution, and growth curves were recorded by periodic measurement at OD₆₆₀ using spectrophotomer (Pharmacia LKB Novaspec II), for determination of NBC (the extract concentration that does not allow bacterial growth after 24 h).

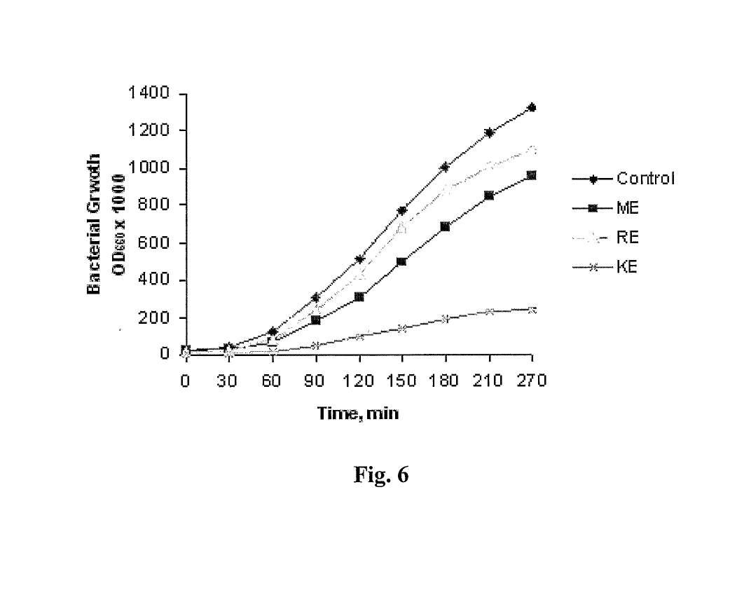

The antibacterial activity of the Balanites saponins was evaluated in a series of concentrations (0.01-1.0% w/v) and a concentration dependent result was obtained. In lower concentration, all saponins showed higher bacterial growth, however, in higher concentration an inhibition of growth was observed. Root purified main saponin (RE) increased the bacterial growth up to 0.75% (w/v) level and mesocarp purified main saponin (ME) increased the bacterial growth only up to 0.5% (w/v) whereas the kernel purified main saponin (KE) showed maximum bacterial growth at a much lower level (0.05% w/v). At 1.0% (w/v), KE achieved the highest growth inhibition (av 80.98%) followed by ME (33.3%) and RE (18.0%). These results, shown in FIG. 6, indicate that Balanites purified saponins from various plant tissues, varying only in their C3 sugar chain, differ in their biological activity.

### REFERENCES

Baccou, J. C. Lambert, F. Sauvaire, Y., “Spectrophotometric method for the determination of total steroidal sapogenin”, Analyst., 1977, 102(1215), 458-465
Cui, M. Song, F. Zhou, Y. Liu, Z. Liu, S., “Rapid identification of saponins in plant extracts by electrospray ionization multi-stage tandem mass spectrometry and liquid chromatography/tandem mass spectrometry”, Rapid Commun. Spectrum, 2000, 14, 1280-1286
Demus, D., Goodby, J. W., Gray, G. W., Spiess, H. W., Vill, V., “Physical properties of liquid crystals”, Wiley-VCH, Weinheim, 1999, ISBN3-527-29747-2
Finsy, R., “Particle sizing by quasi-elastic light scattering”, Adv. Colloid Interface Sci. 1994, 52, 79-143
Hansma, H. G. Laney, D. E., “DNA binding to mica correlates with cationic radius: Assay by Atomic Force Microscopy”, Biophysica J., 1996, 70, 1933-1939
Hosny, M. Khalifa, T. Calis, I. Wright, A. D. Sticher, O., “Balanitoside, a furostanol glycoside, and 6-methyldiosgenin from Balanites aegyptiaca”, Phytochemistry 1992, 31: 3565-3569
Hostettmann, K. Marston, A., “Chemistry and Pharmacology of Natural Products: Saponins”, Cambridge University Press, 1995, 550 p
Ibrahim, A. M., “Anthelmintic activity of some Sudanese medicinal plants”, Phytotherapy Research, 1992, 3, 155-157
Jain, D. C. Tripathi, A. K. “Insect feeding-deterrent activity of saponin glycosides”, Phytotherapy Res. 1991, 5, 139-141
Kamel, M. S., “A furostanol saponin from fruits of Balanites aegyptiaca”, Phytochemistry 1998, 48, 755-757
Kamel, M. S. Ohtani, K. Kurokawa, T. Assaf, M. H. El-Shanawany, M. A. Ali, A. A. Kasai, R. Ishibashi, S. Tanaka, O., “Studies on Balanites aegyptiaca fruits, an antidiabetic Egyptian folk medicine”, Chem Pharm Bull (Tokyo) 1991, 39, 1229-1233
Liang, F. Li, L. J. Abliz, Z. Yang, Y. C. Shi, J. G., “Structural characterization of steroidal saponins by electrospray ionization and fast-atom bombardment tandem mass spectrometry”, Rapid commun Mass spectrum. 2002, 16(12), 1168-1173
Liu, H. W. Nakanishi, K., “The Structures of Balanitins, Potent Molluscicides Isolated from Balanites aegyptiaca”, Tetrahedron, 1982, 38, 513-519
Liu, Y. Liang, F. Cui, L. Xia, M. Zhao, L. Yang, Y. C. Shi, J. G. Abliz, Z., “Multi-stage mass spectrometry of furostanol saponins combined with electrospray ionization in positive and negative ion modes”, Rapid Commun Mass Spectrom. 2004, 18, 235-238

Pettit, G. R. Doubek, D. L. Herald, D. L. Numata, A. Takahasi, C. Fujiki, R. Miyamoto, T., “Isolation and structure of cytostatic steroidal saponins from the African medicinal plant Balanites aegyptica”, J Nat Prod. 1991, 54(6), 1491-1502
Price, K. R. Johnson, I. T. Fenwick, G. R., “The chemistry and biological significance of saponins in foods and feeding-stuffs”, Food Sci. Nutr., 1987, 26, 27-135
Rao, M. V. Shah, K. D. Rajani, M., “Contraceptive efficacy of Balanites roxburghii pericarp extract in male mice (Mus musculus)”, Phytotherapy Research 1997, 11, 469-471
Schonherr, J., “Calcium chloride penetrates plant cuticles via aqueous pores”, Planta 2000, 212, 112-118
Schonherr, J. Riederer, M., “Plant cuticles sorb lipophilic compounds during enzymatic isolation”, Plant Cell Environ. 1986, 9, 459-466
Thomson, N. H. Kasas, S. Smith, B. Hansma, H. G. Hansma, P. K. “Reversible binding of DNA to mica for AFM imaging”, Langmuir 1996, 12, 5905-5908
Tsror, L. Hazanovsky, M. Mordechi-Lebiush, S. Sivan, S., “Aggressiveness of Verticillium dahlia isolates from different vegetative compatibility groups to potato and tomato”, Plant Pathology, 2001, 50, 477-482
Wiesman, Z. Riov, J. Epstein, E., “Comparison of movement and metabolism of indole-3-acetic acid and indole-3-butyric acid in mung bean cuttings”, Physiol Plant 1988, 74, 556-560

1. A saponin of the general formula I: 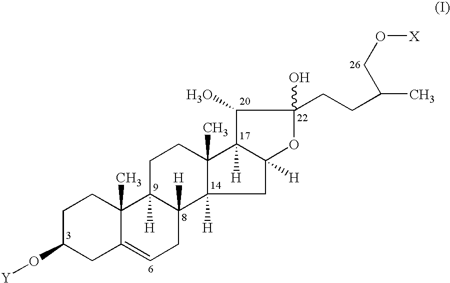
wherein
X is absent or is a glucose residue linked through its C1 position;
Y is absent or is a sugar chain selected from the group consisting of: 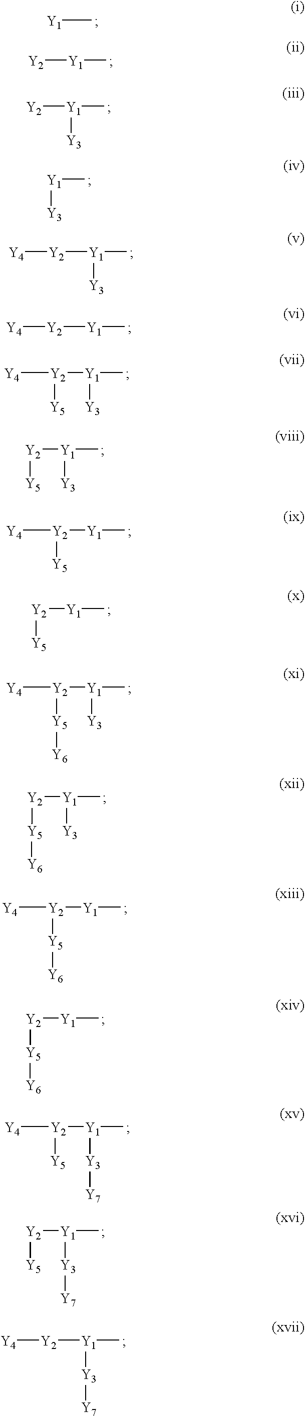 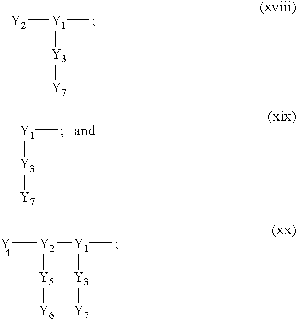
wherein
Y₁ is a glucose residue linked through its C1 position;
Y₂ is a glucose residue linked 1→4 to Y₁;
Y₃ is a rhamnose residue linked 1→2 to Y₁;
Y₄ is a xylose or a rhamnose residue linked 1→3 to Y₂;
Y₅ is a glucose residue linked 1→2 to Y₂;
Y₆ is a glucose residue linked 1→4 to Y₅, and
Y₇ is a glucose residue linked 1→4 to Y₃,
and hydrates and methylated derivatives thereof, but excluding the compound wherein X is a glucose residue and Y is the sugar residue (iii), and mixtures thereof.

2. A saponin of the general formula I in claim 1 and mixtures thereof, wherein said saponins are extracted from the Balanites aegyptiaca fruits (mesocarp or kernel), roots, kernel cake or oil.

3. A stable preparation of Balanites aegyptiaca or Balanites roxburghii saponin nanovesicles encapsulating an active material.

4. The stable preparation according to claim 3, wherein said saponin nanovesicles are formed by mixing saponins or a plant extract rich in saponins in aqueous solution in a concentration that is above the critical micelle concentration.

5. (canceled)

6. The stable preparation according to claim 3, wherein said Balanites aegyptiaca saponin is a saponin of the general formula I or a mixture of at least two different saponins of the general formula I, wherein general formula I is: 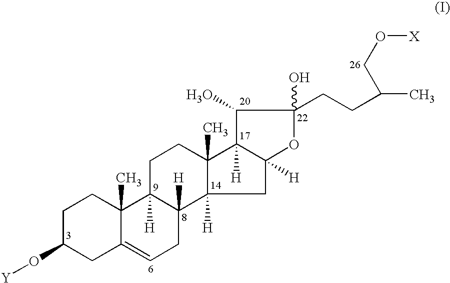
wherein
X is absent or is a glucose residue linked through its C1 position;
Y is absent or is a sugar chain selected from the group consisting of: 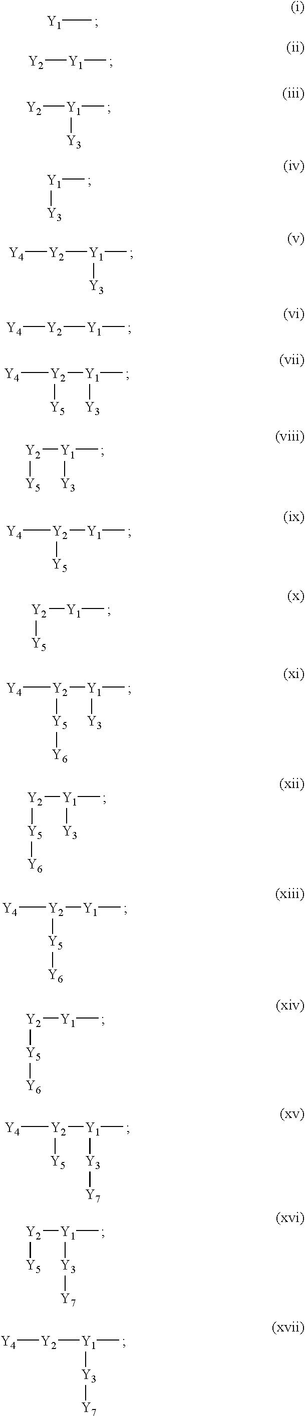 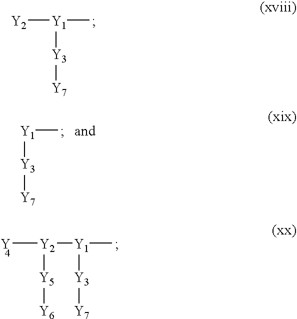
wherein
Y₁ is a glucose residue linked through its C1 position;
Y₂ is a glucose residue linked 1→4 to Y₁;
Y₃ is a rhamnose residue linked 1→2 to Y₁;
Y₄ is a xylose or a rhamnose residue linked 1→3 to Y₂;
Y₅ is a glucose residue linked 1→2 to Y₂;
Y₆ is a glucose residue linked 1→4 to Y₅, and
Y₇ is a glucose residue linked 1→4 to Y₃,
and hydrates and methylated derivatives thereof, but excluding the compound wherein X is a glucose residue and Y is the sugar residue (iii).

7. The stable preparation according to claim 3, wherein said active material is selected from a drug, a toxin, a pesticide, a vitamin, a hormone, a plant growth substance, a mineral, a nutrient, a nucleic acid, an aroma and flavor compound, a flavonoid, a colloid, or a mixture thereof.

8. The stable preparation according to claim 7, wherein said active material is a biopesticide.

9. The stable preparation according to claim 8, wherein said biopesticide is Bacillus thuringiensis Israelensis (Bti) toxin.

10. A pesticidal composition comprising a preparation of Balanites aegyptiaca saponin nanovesicles encapsulating Bti toxin.

11. A method of controlling the growth of mosquito larvae comprising dispersing the pesticidal composition according to claim 10 over agricultural areas affected by said larvae.

12. The stable preparation according to claim 7, wherein said active material is the herbicidal 2,4-dichlorophenoxyacetic acid (2,4-D).

13. The stable preparation according to claim 7, wherein said active material is a vitamin such as vitamin A, B, C, D, E, F, K, P, or derivatives thereof, and mixtures thereof.

14. The stable preparation according to claim 13 comprising a preparation of Balanites aegyptiaca saponin nanovesicles encapsulating said vitamin or mixture thereof.

15. A dermatological or cosmetic composition comprising the stable preparation of claim 14 and a dermatologically or cosmetically acceptable carrier.

16. The dermatological or cosmetic composition according to claim 15 comprising vitamin C encapsulated within Balanites aegyptiaca saponin nanovesicles.

17-20. (canceled)

21. An agricultural composition for foliar and/or soil application comprising an agrochemical and a saponin-rich plant extract.

22. The agricultural composition according to claim 21 wherein said agrochemical is a nutrient, a plant growth regulator, or a pesticide.

23. The agricultural composition according to claim 22 wherein said agrochemical is an herbicide, preferably 2,4-D.

24. The agricultural composition according to claim 21 comprising saponin-rich extract from Quillaja saponaria or B. aegyptiaca.

25-26. (canceled)

27. A method of enhancing the rooting of cuttings comprising applying to the bases of the cuttings an aqueous solution of B. aegyptiaca saponins, optionally together with the plant hormone indole-3-butyric acid (IBA).

28. A method of enhancing the germination of crops seeds and the development of germinated seedlings irrigated with low quality saline water, comprising adding B. aegyptiaca saponins to the irrigation saline water.

29. A method of inhibiting the growth of mycelial colonies affecting plants, comprising dispersing over agricultural areas an aqueous solution of B. aegyptiaca saponins.

30. The method according to claim 27 wherein the saponin is a saponin of the general formula I or a mixture of at least two different saponings of general formula I, wherein general formula 1 is: 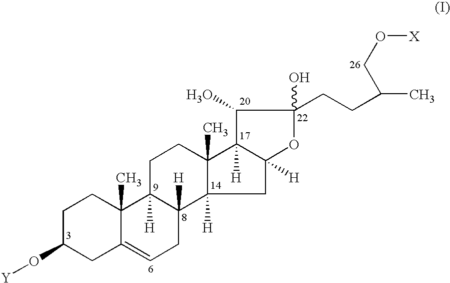
wherein
X is absent or is a glucose residue linked through its C1 position;
Y is absent or is a sugar chain selected from the group consisting of: 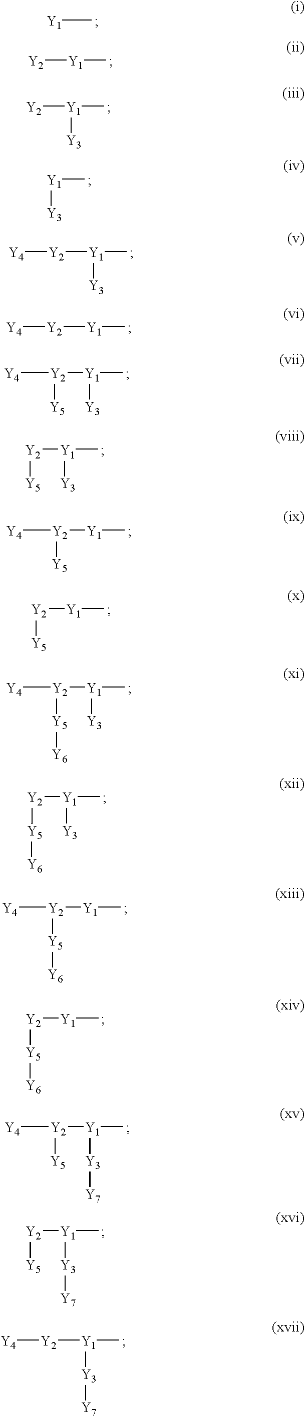 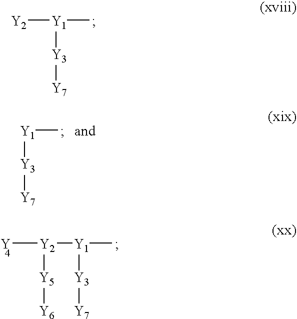
wherein
Y₁ is a glucose residue linked through its C1 position;
Y₂ is a glucose residue linked 1→4 to Y₁;
Y₃ is a rhamnose residue linked 1→2 to Y₁;
Y₄ is a xylose or a rhamnose residue linked 1→3 to Y₂;
Y₅ is a glucose residue linked 1→2 to Y₂;
Y₆ is a glucose residue linked 1→4 to Y₅, and
Y₇ is a glucose residue linked 1→4 to Y₃,
and hydrates and methylated derivatives thereof, but excluding the compound wherein X is a glucose residue and Y is the sugar residue (iii).

31. (canceled)

32. A method of extending life and efficiency of a petroleum fuel or biofuel based engine, which comprises adding saponins or a saponin-rich plant extract such as B. aegyptiaca saponins or extract to the petroleum fuel of biofuel consumed by said engine.

33. A saponin of the general formula I in claim 1 wherein X is absent or is a glucose residue linked through its C1 position, and Y is a sugar chain (i), (ii), (iv), (v), (vi), (vii), (viii), (ix), (x), (xi), (xii), (xiii), (xiv), (xv), (xvi), (xvii), (xviii), (xix) or (xx).

34. The method according to claim 28 wherein the saponin is a saponin of the general formula I or a mixture of at least two different saponins of the general formula I, wherein general formula I is: 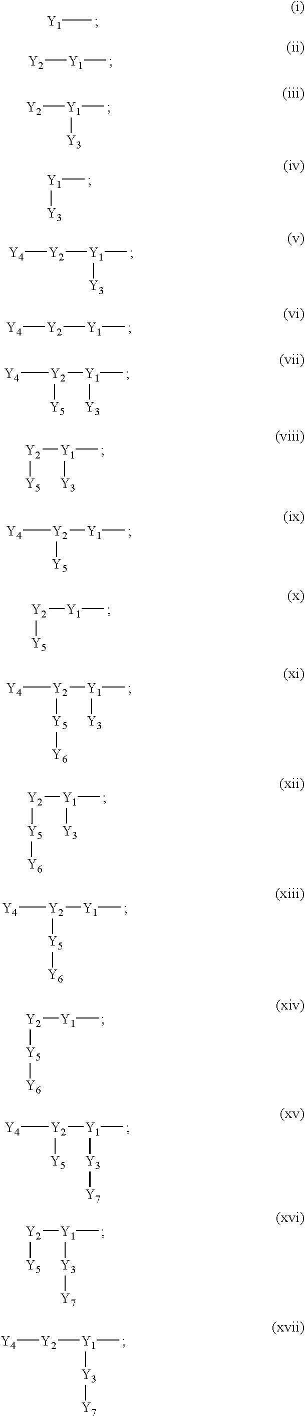 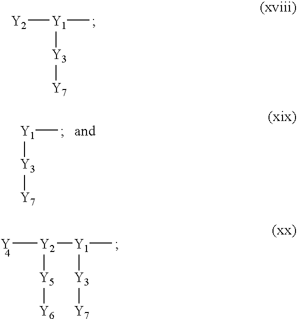
wherein
Y₁ is a glucose residue linked through its C1 position;
Y₂ is a glucose residue linked 1→4 to Y₁;
Y₃ is a rhamnose residue linked 1→2 to Y₁;
Y₄ is a xylose or a rhamnose residue linked 1→3 to Y₂;
Y₅ is a glucose residue linked 1→2 to Y₂;
Y₆ is a glucose residue linked 1→4 to Y₅, and
Y₇ is a glucose residue linked 1→4 to Y₃,
and hydrates and methylated derivatives thereof, but excluding the compound wherein X is a glucose residue and Y is the sugar residue (iii).

35. The method according to claim 29 wherein the saponin is a saponin of the general formula I or a mixture of at least two different saponins of the general formula I, wherein general formula I is: 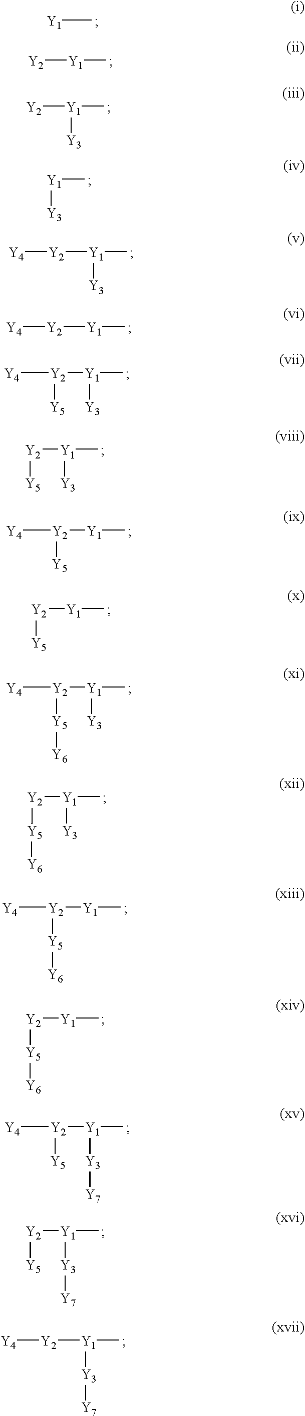 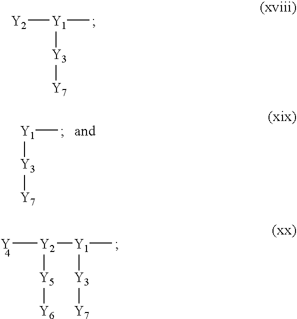
wherein
Y₁ is a glucose residue linked through its C1 position;
Y₂ is a glucose residue linked 1→4 to Y₁;
Y₃ is a rhamnose residue linked 1→2 to Y₁;
Y₄ is a xylose or a rhamnose residue linked 1→3 to Y₂;
Y₅ is a glucose residue linked 1→2 to Y₂;
Y₆ is a glucose residue linked 1→4 to Y₅, and
Y₇ is a glucose residue linked 1→4 to Y₃,
and hydrates and methylated derivatives thereof, but excluding the compound wherein X is a glucose residue and Y is the sugar residue (iii).
